(19) United States
(12) Patent Application Publication
(10) Pub. No.: US 2024/0035933 A1
(43) Pub. Date: Feb. 1, 2024

(54) FLUID SENSOR CARTRIDGE

(71) Applicant: Analog Devices, Inc., Wilmington, MA (US)

(72) Inventors: David Frank Bolognia, Charlestown, MA (US); Erin Eda Evke, Boston, MA (US)

(21) Appl. No.: 18/361,750

(22) Filed: Jul. 28, 2023

Related U.S. Application Data
(60) Provisional application No. 63/370,089, filed on Aug. 1, 2022.

(51) Int. Cl. G01N 1/20; G01N 33/49

(52) U.S. Cl. CPC G01N 1/2035; G01N 33/49; G01N 2001/205; G01N 2001/1427

(57) ABSTRACT

A fluid sensor cartridge that can be used to monitor composition of constituent materials or characteristics of a sample fluid or a patient's blood. The fluid sensor cartridge can be connected in-line with a medical device during a medical treatment. The fluid sensor cartridge can utilize one or more valves, one or more channels, and one or more reservoirs to enable sampling, sensing, and calibrating of a sample fluid or a patient's blood in one container with minimal amounts of sample/blood and calibration fluid.

### CROSS-REFERENCE TO RELATED APPLICATION

This application claims the benefit of U.S. Provisional Application No. 63/370,089, entitled FLUID SENSOR CARTRIDGE, and filed on Aug. 1, 2022. U.S. Provisional Application No. 63/370,089 is incorporated by reference herein.

### BACKGROUND

### Field

The field relates to a fluid sensor cartridge.

### Description of the Related Art

Treatment procedures, such as dialysis procedures, may need to be performed on a regular basis in a hospital setting or outpatient facility. It can be desirable to monitor the constituent materials of the patient's blood and/or a treatment fluid during the treatment procedures in real-time. Blood or fluid tests in the lab or with one-time use card-style sensors before or after the treatment can be inaccurate, inconvenient, time-consuming, and economically costly to the patient and the clinician. Enabling such real time testing and monitoring to be performed with a small cartridge that can be connected in-line to a medical device can advantageously improve the accuracy, convenience, efficiency, and affordability of the procedures.

### SUMMARY

In one embodiment, a fluid cartridge sensor described herein comprises a sensor assembly including a fluid pathway and one or more sensing elements in fluid communication with the fluid pathway; a first fluid inlet configured to provide a first sample fluid to the fluid pathway; a first valve configured to selectively direct the first sample fluid to the fluid pathway; a second fluid inlet configured to provide a second fluid to the fluid pathway; and a second valve configured to selectively direct the second fluid to the fluid pathway.

In some aspects, the fluid sensor cartridge further includes a first drain in fluid communication with the fluid pathway, the first drain to convey fluid from the fluid pathway to a waste reservoir.

In some aspects, the fluid cartridge sensor includes a container including a calibration fluid reservoir to hold a calibration fluid and a waste reservoir; a sensor assembly including a fluid pathway and one or more sensing elements in fluid communication with the fluid pathway; a first fluid inlet configured to provide a first sample fluid to the fluid pathway; a second fluid inlet configured to provide at least a second fluid to the fluid pathway; and a first drain in fluid communication with the fluid pathway, the first drain to convey fluid from the fluid pathway to the waste reservoir.

In some aspects, the fluid sensor cartridge further includes a first valve configured to selectively direct the first sample fluid to the fluid pathway and a second valve configured to selectively direct the second fluid to the fluid pathway.

In some aspects, the first valve includes a rotatable plug valve having a plurality of operating positions.

In some aspects, the second valve includes a face valve including a plate having a plurality of openings, the face valve rotatable relative to the first and second fluid inlets.

In some aspects, the fluid sensor cartridge further includes a plunger configured to control flow of fluid in the fluid pathway.

In some aspects, the fluid sensor cartridge further includes a third valve configured to fluidly connect the fluid pathway to the plunger.

In some aspects, the third valve includes a second rotatable plug valve having a plurality of operating positions.

In some aspects, the fluid sensor cartridge further includes a second drain spaced apart from the first drain, wherein the plurality of openings includes a setup mode fluid inlet opening and a setup mode drain opening, wherein during a setup mode of the fluid sensor cartridge, the setup mode fluid inlet opening at least partially overlaps with the second fluid inlet, the setup mode drain opening at least partially overlaps with the second drain, and the first drain is occluded.

In some aspects, wherein in the setup mode, the first valve is in a closed position to inhibit fluid flow into or out of the fluid pathway through the first valve.

In some aspects, the plurality of openings includes a calibration mode fluid inlet opening and a calibration mode drain opening, wherein during a calibration mode of the fluid sensor cartridge, the calibration mode fluid inlet at least partially overlaps with the second fluid inlet and the calibration mode drain opening at least partially overlaps with the first drain.

In some aspects, the calibration mode fluid inlet opening and the calibration mode drain opening are disposed at generally opposite circumferential positions of the plate.

In some aspects, wherein in the calibration mode, the first valve is in a closed position to inhibit fluid flow into or out of the fluid pathway through the first valve.

In some aspects, the plurality of openings includes a quality control (QC) mode fluid inlet opening and a QC mode drain opening, wherein during a QC mode of the fluid sensor cartridge, the QC mode fluid inlet opening at least partially overlaps the second fluid inlet and the QC mode drain opening at least partially overlaps the first drain.

In some aspects, the plurality of openings includes a plurality of QC mode fluid inlet openings and a plurality of QC mode drain openings, the fluid sensor cartridge including a plurality of QC operating modes.

In some aspects, wherein in the QC mode, the first valve is in a closed position to inhibit fluid flow into or out of the fluid pathway through the first valve.

In some aspects, the fluid sensor cartridge further includes a third drain to convey fluid to the waste reservoir, wherein the plurality of openings includes a valve drain opening, wherein in a valve flush mode of the fluid sensor cartridge, the valve drain opening at least partially overlaps the third drain, the third drain disposed below the first valve.

In some aspects, wherein in the valve flush mode, the first valve is at least partially open to permit a calibration fluid to flush the first valve to the waste reservoir.

In some aspects, wherein in the valve flush mode, the first drain is occluded.

In some aspects, the fluid sensor cartridge has an inflow mode in which the first valve is at least partially open to provide the sample fluid to the fluid pathway.

In some aspects, the plurality of openings includes a sample fluid drain opening, and, wherein, in the inflow mode, the sample fluid drain at least partially overlaps with the first drain.

In some aspects, the fluid sensor cartridge has a sense mode in which the sensor assembly transduces one or more fluid properties of the sample fluid in the fluid pathway.

In some aspects, in the sense mode, the first drain is occluded.

In some aspects, the second valve further includes a gasket disposed over the plate of the second valve, the gasket including a plurality of gasket openings, the second valve rotatable relative to the gasket.

In some aspects, the fluid sensor cartridge further includes a manifold over the gasket, the manifold including the first fluid inlet, the second fluid inlet, and the first drain.

In some aspects, the first valve is disposed in an opening of the manifold.

In some aspects, the fluid sensor cartridge further includes a wicking pad disposed in an aperture of the gasket.

In some aspects, the wicking pad is configured to selectively occlude and expose the second fluid inlet upon rotation of the second valve.

In some aspects, the sensor assembly includes a plurality of first electrodes exposed to the fluid pathway.

In some aspects, the sensor assembly further includes a second electrode spaced apart from the plurality of first electrodes.

In some aspects, the second electrode is disposed along a second fluid pathway spaced from the fluid pathway, an angled segment fluidly connecting the first and second fluid pathways.

In some aspects, the angled segment and the second fluid pathways are configured to maintain fluid in the second fluid pathway.

In some aspects, the fluid sensor cartridge further includes a container, the face valve disposed at a first end portion of the container.

In some aspects, the fluid sensor cartridge further includes a calibration fluid reservoir in the container and a calibration fluid plunger configured to drive the calibration fluid along a calibration channel of the container and out of the container through a calibration fluid inlet opening of the plate.

In some aspects, the fluid sensor cartridge further includes a waste reservoir in communication with at least one drain opening of the plate.

In some aspects, the waste container is disposed around the calibration reservoir.

In some aspects, the fluid sensor cartridge further includes a plurality of reservoirs in the container and a corresponding plurality of plungers, each configured to drive a corresponding fluid and out of the container through a corresponding fluid inlet opening of the plate.

In some aspects, the fluid sensor cartridge further includes a plurality of straws, each disposed inside each of the plurality of reservoirs, a corresponding fluid flowing through the straws and through a corresponding fluid inlet opening of the plate.

In some aspects, the plurality of reservoirs includes a plurality of QC reservoirs containing a corresponding plurality of QC fluids.

In some aspects, the fluid sensor cartridge further includes a mechanical interface at a second end portion of the container, the mechanical interface configured to removably couple to a reader assembly which is configured to impart rotation to the mechanical interface and the container.

In some aspects, the mechanical interface includes a gear.

In some aspects, the fluid sensor cartridge further includes an electrical interface electrically connected to the sensor assembly, the electrical interface configured to removably couple to the reader assembly.

In some aspects, the fluid sensor cartridge further includes the reader assembly.

In some aspects, the reader assembly includes a controller configured to drive one or more plungers to move the second fluid to the fluid pathway.

In some aspects, the controller is configured to automatically switch between a plurality of operating modes.

In some aspects, the plurality of operating modes includes at least two of a setup mode, a sample inflow mode, a sense mode, a calibration mode, one or more valve flush modes, and a quality control (QC) mode.

In some aspects, the fluid sensor cartridge further includes tubing in fluid communication with the first inlet, the tubing configured to connect with a medical device.

In some aspects, the fluid sensor cartridge further includes one or more air inlets in fluid communication with the fluid pathway.

In some aspects, the one or more air inlets are configured to fluidly connect to ambient air.

In some aspects, the one or more air inlets includes a vacuum port configured to pull from a reservoir to overcome a negative pressure of the first sample fluid.

In some aspects, the each air inlet is configured to at least partially overlap with the second fluid inlet, the plunger pulling air from ambient air through the second fluid inlet to the fluid pathway.

In some aspects, the fluid sensor cartridge further includes one or more cavities in the container exposed to ambient air, wherein the one or more air inlets are disposed on the cavities.

In another embodiment, a fluid sensing method includes opening a first plug valve to provide a first sample fluid to a fluid pathway to expose a sensor assembly to at least the first sample fluid; transducing at least one fluid property of the first sample fluid with the sensor assembly; and rotating a second face valve to provide a second fluid to the fluid pathway to flush the sensor assembly, the second fluid flowing along the fluid pathway through a first drain to a waste reservoir.

In some aspects, the method further includes, before transducing, closing the first plug valve and rotating the second face valve to occlude the first drain.

In some aspects, the method further includes rotating the second face valve to provide a third fluid to the fluid pathway, the third fluid flowing along the fluid pathway through the first drain, the third fluid including a quality control (QC) fluid.

In some aspects, the method further includes rotating the second face valve to provide a plurality of fluids to the fluid pathway through a plurality of corresponding openings, each of the one or more fluids flowing along the fluid pathway through the first drain.

In some aspects, the method further includes at least partially opening the first plug valve, and rotating the second face valve to provide the second fluid to the first plug valve to flush the first plug valve, the second fluid flowing through the first plug valve through a valve drain to the waste reservoir.

In some aspects, the method further includes rotating a third plug valve to fluidly connect the fluid pathway to a plunger and controlling flow of fluid in the fluid pathway with the plunger.

In some aspects, the method, further including rotating the third plug valve to provide the fluid in the fluid pathway to flow through the third plug valve through the first drain to the waste reservoir.

In some aspects, the method further includes, before transducing and when the fluid in the fluid pathway is a mixture of the sample fluid and another fluid, closing the third plug valve to the fluid pathway, controlling flow of the mixture of fluid between the third plug valve and the plunger through the third plug valve through the first drain to a waste reservoir with the plunger, rotating the third plug valve to fluidly connect the fluid pathway to the plunger, controlling a flow of sample fluid to the fluid pathway with the plunger, and repeating the process until the fluid in the fluid pathway includes substantially of only the sample fluid.

In some aspects, the method further includes rotating the second face valve and rotating the third plug valve to provide the second fluid to the third plug valve to flush the third plug valve and the plunger, the second fluid flowing through the third plug valve to the plunger; and rotating the third plug valve and pushing the plunger to drive the fluid between the third plug valve and the plunger through the first drain to the waste reservoir.

In some aspects, the method further includes pulling air from one or more cavities open to ambient air to introduce air bubbles to fluid in the fluid pathway.

### BRIEF DESCRIPTION OF THE DRAWINGS

The present disclosure is described with reference to the accompanying drawings, in which like reference characters reference like elements, and wherein:

FIG. 1 is a block diagram illustrating a fluid sensor cartridge according to an embodiment of the present disclosure, shown with a reader assembly.

FIG. 2A is top, front perspective view of the fluid sensor cartridge of FIG. 1.

FIG. 2B is a bottom, rear perspective view of the fluid sensor cartridge of FIG. 2A.

FIG. 3 is a top view of the fluid sensor cartridge of FIG. 2A.

FIG. 4A is a top view of the fluid sensor cartridge of FIG. 2A shown without a manifold and a tubing.

FIG. 4B is a top view of the fluid sensor cartridge of FIG. 4A, shown without a sensor assembly.

FIG. 5A is a top perspective view of the fluid sensor cartridge of FIG. 4B shown without a gasket and a wicking pad.

FIG. 5B is a top view of the fluid sensor cartridge of FIG. 5A.

FIG. 6A is a bottom, rear perspective view of the fluid sensor cartridge of FIG. 2A showing a container as transparent.

FIG. 6B is a bottom, left side perspective view of the fluid sensor cartridge of FIG. 6A.

FIG. 7A is a cross-sectional view of the fluid sensor cartridge of FIG. 3, taken along line A-A.

FIG. 7B is a close-up view of section B of the fluid sensor cartridge of FIG. 6A.

FIG. 8 is a chart showing inlets that are open and closed during each operating mode.

FIG. 9A is a top view of the fluid sensor cartridge of FIG. 2A in a set (“SET”) mode, showing only the manifold and a face valve.

FIG. 9B is a top view of the fluid sensor cartridge of FIG. 5B in a SET mode.

FIG. 10A is a top view of the fluid sensor cartridge of FIG. 4A in a SET mode and with the tubing.

FIG. 10B is a close-up view of section C of the fluid sensor cartridge of FIG. 10A in a SET mode.

FIG. 11A is a top view of the fluid sensor cartridge of FIG. 2A in a fluid in (“IN”) mode, showing only the manifold and a face valve.

FIG. 11B is a top view of the fluid sensor cartridge of FIG. 5B in an IN mode.

FIG. 12 is a top view of the fluid sensor cartridge of FIG. 4A in an IN mode and with the tubing.

FIG. 13A is a top view of the fluid sensor cartridge of FIG. 2A in a sense (“SNS”)/ship mode, showing only the manifold and a face valve.

FIG. 13B is a top view of the fluid sensor cartridge of FIG. 2A in a SNS/ship mode, showing only the manifold and a face valve.

FIG. 14 is a top view of the fluid sensor cartridge of FIG. 4A in a SNS/ship mode and with the tubing.

FIG. 15A is a top view of the fluid sensor cartridge of FIG. 2A in a calibration (“CAL”) mode, showing only the manifold and a face valve.

FIG. 15B is a top view of the fluid sensor cartridge of FIG. 2A in a CAL mode, showing only the manifold and a face valve.

FIG. 16 is a top view of the fluid sensor cartridge of FIG. 4A in a CAL mode and with the tubing.

FIG. 17A is a top view of the fluid sensor cartridge of FIG. 2A in a valve flush (“VLV”) mode, showing only the manifold and a face valve.

FIG. 17B is a top view of the fluid sensor cartridge of FIG. 2A in a VLV mode, showing only the manifold and a face valve.

FIG. 18A is a top view of the fluid sensor cartridge of FIG. 4A in a VLV mode and with the tubing.

FIG. 18B is a close-up view of section D of the fluid sensor cartridge of FIG. 18A in a VLV mode.

FIG. 19A is a top view of the fluid sensor cartridge of FIG. 2A in a quality control (“QC”) mode, showing only the manifold and a face valve.

FIG. 19B is a top view of the fluid sensor cartridge of FIG. 2A in a QC mode, showing only the manifold and a face valve.

FIG. 20 is a top view of the fluid sensor cartridge of FIG. 4A in a QC mode and with the tubing.

FIG. 21A is a top view of the fluid sensor cartridge of FIG. 4A shown without a sensor assembly, with the wicking pad in an open position.

FIG. 21B is a cross-sectional view of the fluid sensor cartridge of FIG. 21A taken along line E-E.

FIG. 22A is a top view of the fluid sensor cartridge of FIG. 21A, with the wicking pad in a closed position.

FIG. 22B is a cross-sectional view of the fluid sensor cartridge of FIG. 22A taken along line F-F.

FIG. 23A is a side plan view of the fluid sensor cartridge of FIG. 22B, with the wicking pad in an open position.

FIG. 23B illustrate the fluid sensor cartridge of FIG. 22B with the wicking pad in a closed position.

FIG. 24 is a top, front perspective view of another embodiment of a fluid sensor cartridge according to this disclosure, showing a manifold as transparent.

FIG. 25A is a front view of the fluid sensor cartridge of FIG. 24, showing a plunger pushed up.

FIG. 25B is a front view of the fluid sensor cartridge of FIG. 24, showing the plunger pulled down.

FIG. 26 is a top view of the fluid sensor cartridge of FIG. 24, showing the manifold as sold.

FIG. 27 is a top view of the fluid sensor cartridge of FIG. 24.

FIG. 28A is a top view of the fluid sensor cartridge of FIG. 27 in a first step of an IN mode.

FIG. 28B is a top view of the fluid sensor cartridge of FIG. 27 in a second step of the IN mode.

FIG. 28C is a top view of the fluid sensor cartridge of FIG. 27 in a third step of the IN mode.

FIG. 28D is a close-up view of section G of the fluid sensor cartridge of FIGS. 28A and 28C.

FIG. 28E is a close-up view of section H of the fluid sensor cartridge of FIG. 28B.

FIG. 29 is a top view of the fluid sensor cartridge of FIG. 24 in a CAL mode.

FIG. 30A is a top view of the fluid sensor cartridge of FIG. 24 in a first step of a plunger flush (PLG) mode.

FIG. 30B is a top view of the fluid sensor cartridge of FIG. 24 in a second step of the PLG mode.

FIG. 31A is a side view of the fluid sensor cartridge of FIG. 24 with a container shown as transparent.

FIG. 31B is a top view of the fluid sensor cartridge of FIG. 24 with a manifold shown as transparent in a QC mode.

FIG. 32A is a bottom perspective view of the fluid sensor cartridge of FIG. 31A with the container shown as solid.

FIG. 32B is a top cross-sectional view of the fluid sensor cartridge of FIG. 31A, taken along line I-I.

FIG. 32C is a top view of the fluid sensor cartridge of FIG. 31B in an air intake mode.

### DETAILED DESCRIPTION

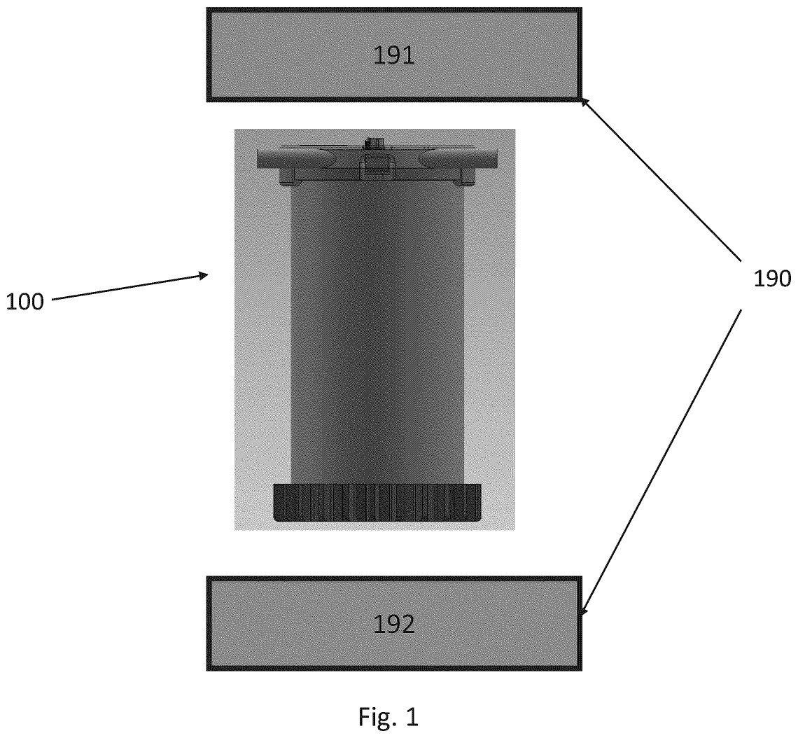

Various embodiments relate herein to a fluid sensor cartridge configured to be connected in-line to a medical device, such as a dialysis treatment system. FIGS. 1-22B show an embodiment of the fluid sensor cartridge 100 and a plurality of modes the fluid sensor cartridge 100 can be operated in. In some embodiments, the fluid sensor cartridge 100 can include a tubing 102 configured to fluidly connect to a treatment system of the medical device. For example, in kidney hemodialysis systems, blood can be transferred from the patient, through the dialysis treatment system, and back in the patient, to treat the patient's blood. In a peritoneal dialysis (PD) system, the treatment system can pump dialysate in a circulation through the abdominal cavity. During a treatment procedure, it can be important to monitor the composition of constituent materials in the patient's blood, such as creatinine, potassium, sodium, or any other constituent material that should be monitored and/or the composition of constituent materials in a sample fluid, such as a treatment fluid (e.g., dialysate). In addition, it can also be important to monitor characteristics (e.g. pH, conductivity, etc.) of the patient's blood or that of a sample fluid. The fluid sensor cartridge 100 can be placed upstream or downstream of the treatment system or a filter to monitor the constituent materials in a sample fluid 10.

In some embodiments, the fluid sensor cartridge 100 can include a lid 104 coupled to the manifold 110 to protect components on the fluid sensor cartridge 100. In some embodiments, the lid 104 can be transparent or semi-transparent so that a fluid inside the fluid pathway can is visible to a user.

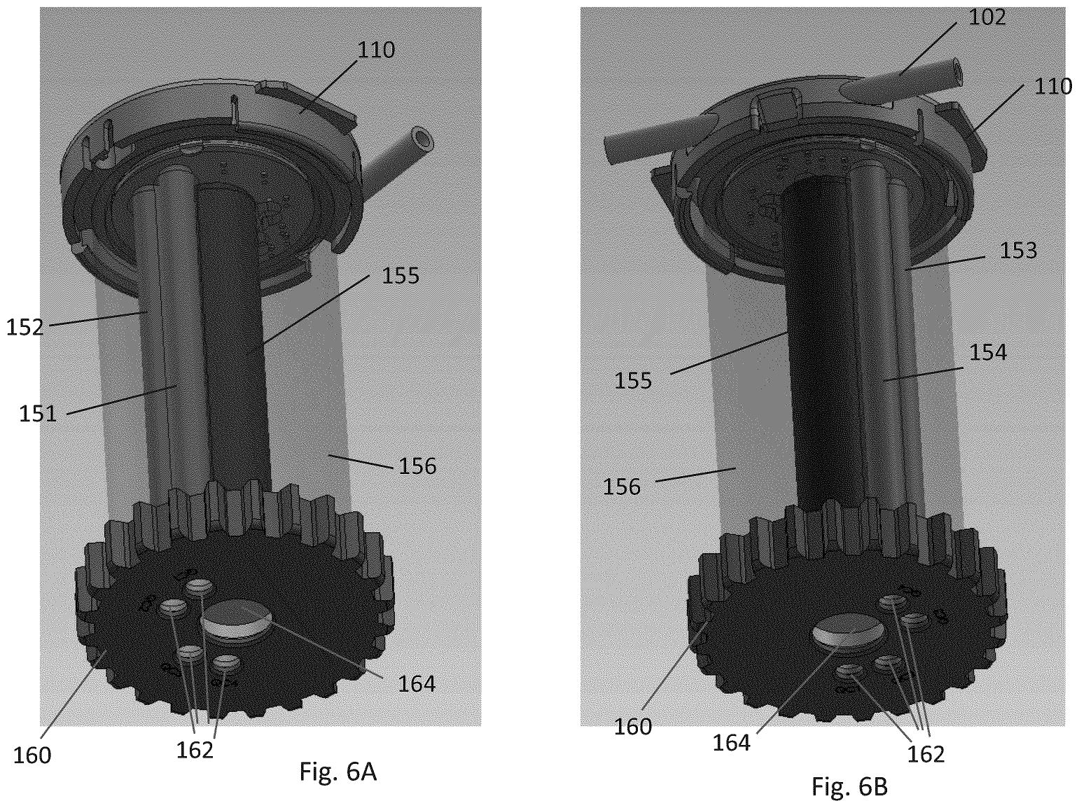

Beneficially, the fluid sensor cartridge 100 can be sized and configured to accurately monitor multiple different constituents in a sample at the same time and require minimal amount of sample (e.g. blood or treatment fluid) to complete a test. For example, as shown in FIGS. 6A-6B, the fluid sensor cartridge 100 can have a container 150 comprising multiple reservoirs to hold a plurality of quality control (QC) fluids and a calibration fluid 20. In some embodiments, the multiple reservoirs can include a first QC reservoir 151, a second QC reservoir 152, a third QC reservoir 153, a fourth QC reservoir 154, and a calibration reservoir 155. In some embodiments, the container 150 can also include a reservoir filled with air or any other kind of fluids to fit different needs. The capability of holding and testing with a plurality of QC/calibration fluids enables accurate testing of multiple different constituents in a sample and testing constituents for many times over multiple days. Further, the fluid pathway can be configured to be of a minimal length to reduce the sample size required to complete a test.

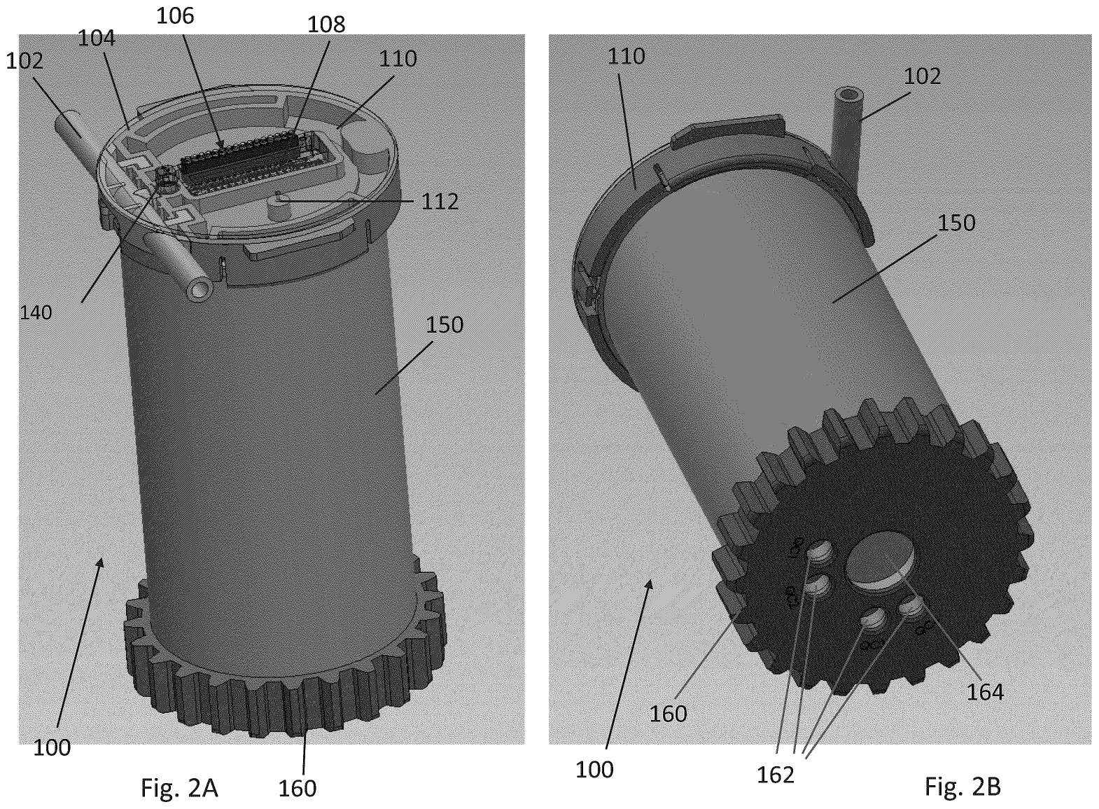

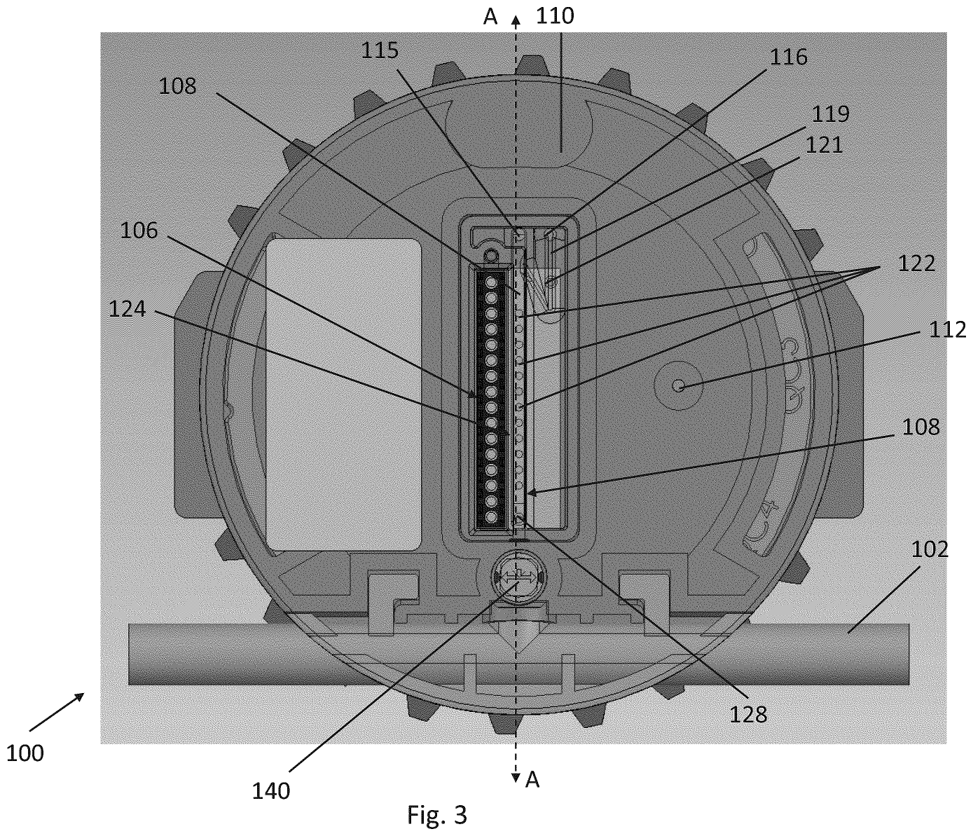

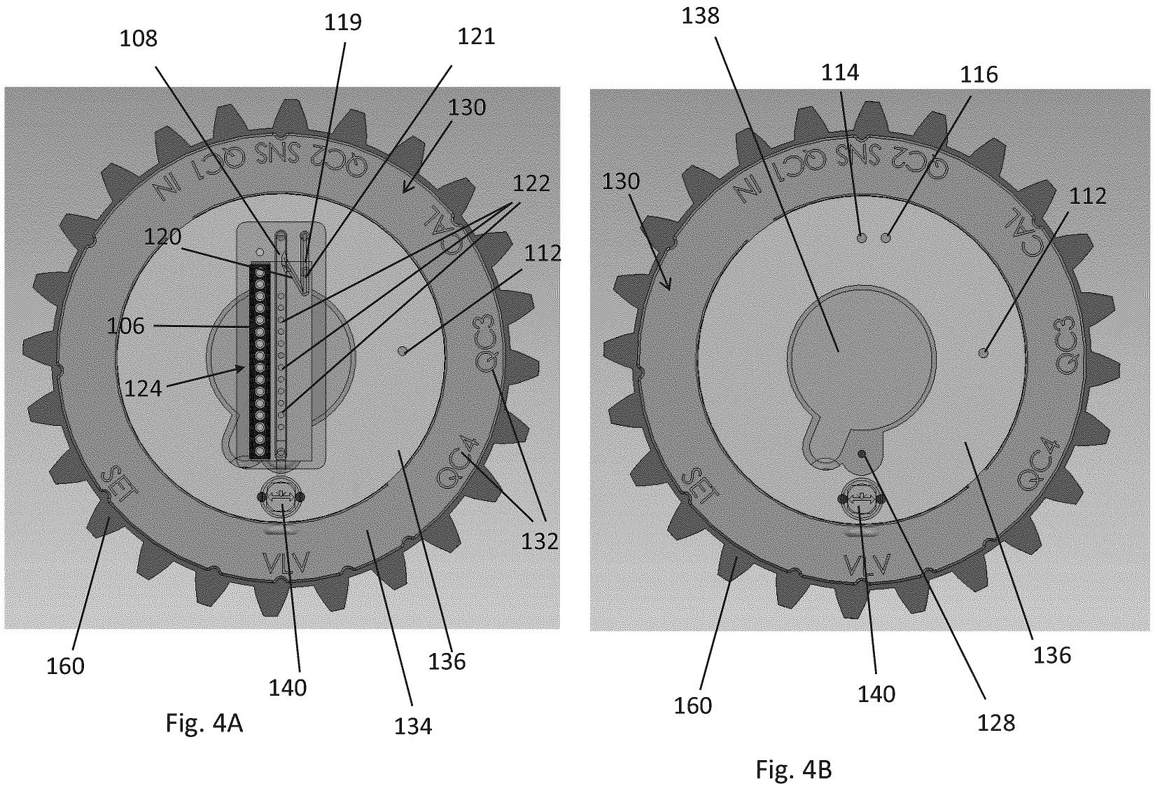

In some embodiments, the fluid sensor cartridge 100 can further comprise a manifold 110 coupled to the container 150 as shown in FIG. 2A-2B. The manifold 110 can hold a main fluid pathway 108 and one or more sensing elements. In some embodiments, the one or more sensing elements can comprise a plurality of primary electrodes 122 (e.g. ion and/or metabolite sensors) that, when exposed to the sample fluid 10, transmit one or more signals indicative of one or more constituent components of the sample fluid 10. As shown, the main fluid pathway 108 can extend over the primary electrodes 122 to expose the primary electrodes 122 to the sample fluid 10. In some embodiments, the manifold 110 can also include a secondary fluid pathway 119 fluidly connected to the main fluid pathway 108 by an angled sideway 120 (see FIG. 3-4A). A fluid (e.g. calibration fluid 20, QC fluids 30, or the sample fluid 10) can be maintained in the secondary fluid pathway 119 due to the angle of the angled sideway 120, such that, during operation of the fluid sensor cartridge 100, the fluid can be maintained over the secondary fluid pathway 119. In some embodiments, the one or more sensing elements can further include one or more secondary electrodes 121 positioned in the secondary fluid pathway 119 and exposed to the fluid in the secondary fluid pathway 119. In some embodiments, the one or more sensing elements can all be positioned on a sensor assembly 124.

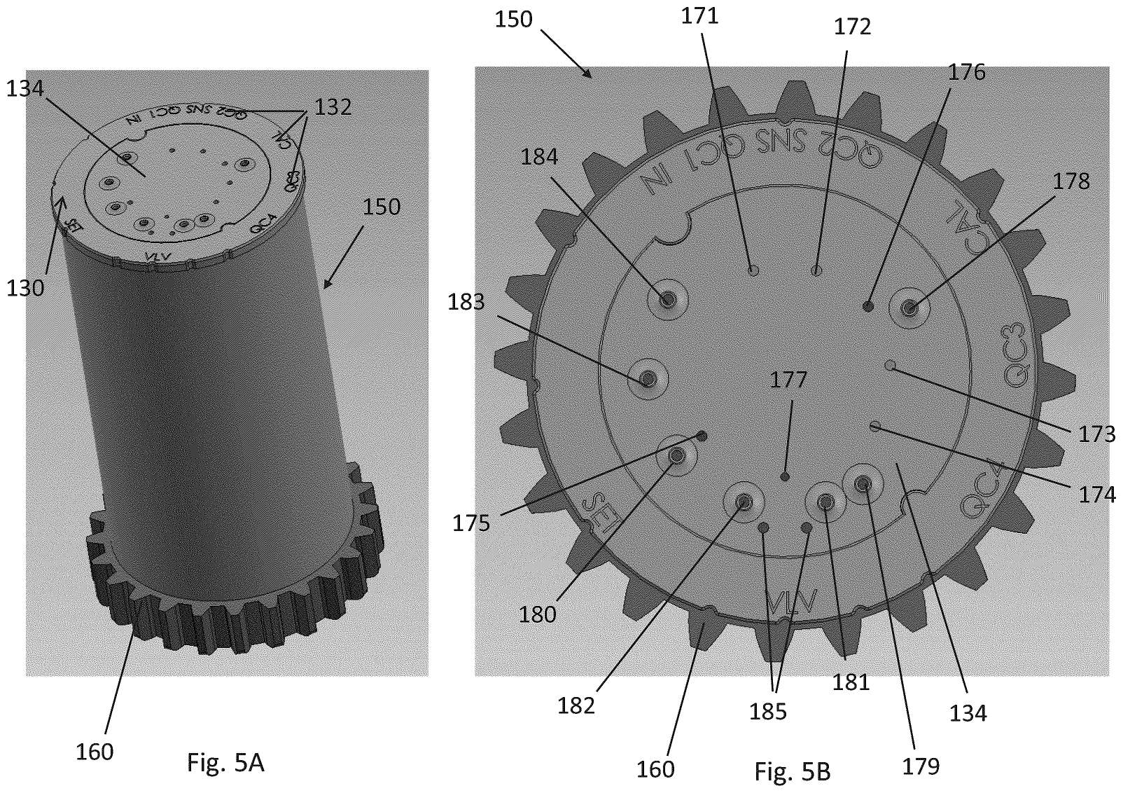

As shown in FIG. 3, the fluid sensor cartridge 100 can include a plug valve 140 coupled to the manifold 110 and configured to switch between a plurality of positions including an open position O allowing the sample fluid 10 to flow in via a sample fluid inlet 129 and a closed position X closed to the tubing 102. In some embodiments, the plug valve 140 can be switched between the plurality of positions by rotating a knob coupled to the plug valve 140. In some embodiments, the fluid sensor cartridge 100 can further include a face valve 130 coupled to the container 150 and include a plurality of drains or inlet openings as shown in FIG. 4A-5B. The face valve 130 can be configured to switch between a plurality of operational modes by aligning openings of the reservoirs to the drain holes or inlets on the manifold 110. In some embodiments, in each operating mode, the face valve 130 can include a corresponding mode indicia 132, visible through an opening on the manifold 110. In some embodiments, the face valve 130 can include a gasket 136 disposed over and configured to rotate relative to a plate of the face valve 134 (“face plate 134”) to occlude or leave open the openings on the face valve 130. The fluid sensor cartridge 100 can thereby feed the plurality of QC fluids 30/calibration fluid 20 to the main fluid pathway 108 through the same QC/calibration fluid inlet opening 128 on the manifold 110. After use, the fluids (e.g. QC, calibration, and sample fluids) can all go to a waste reservoir 156 through their respective drains openings.

Also beneficially, the fluid sensor cartridge 100 can be sized and configured to be used as disposable in a clinical setting such as a hospital or clinic, or at home by the patient, requiring minimal efforts by the user. For example, the QC/calibration fluid inlet opening 128 on the manifold 110 can connect to the treatment system by way of a quick connection such as a Luer lock or other fluid coupling. In some embodiments, the fluid sensor cartridge 100 can mechanically and electrically connect to a reader assembly 190 as shown in FIG. 1. The reader assembly 190 can mechanically couple to the fluid sensor cartridge 100 on one end via a mechanical interface 192. The mechanical interface 192 can comprise a motor and a gear 160 (see. FIG. 2A) that is coupled to the face valve 130 and configured to rotate the face valve 130, in cooperation with the plug valve 140, to achieve different the operating modes. In some embodiments, the reader assembly 190 can electrically couple to the fluid sensor cartridge 100 on another end via an electrical interface 191. In some embodiments, the sensor assembly 124 on the manifold 110 of the fluid sensor cartridge 100 can include a connector 106 to allow the 124 be connected to the electrical interface 191 of the reader assembly 190. In some embodiments, the reader assembly 190 can further comprise a controller configured to drive one or more plungers to move fluids to the main fluid pathway 108. In some embodiments, each reservoir has a corresponding plunger attached to it to push or pull the fluid inside the reservoir. The controller can also be configured to control the face valve 130, in cooperation with operation of the plug valve 140, to automatically switch between the plurality of operating modes.

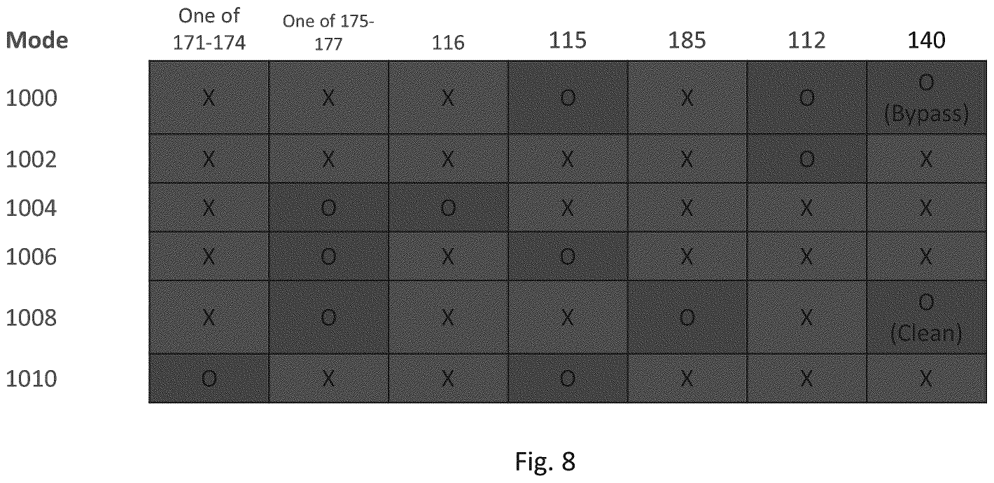

In some embodiments, the face valve 130 can include one or more QC inlet (e.g., four QC inlets 171-174), one or more calibration fluid inlet (e.g., three calibration fluid inlets 175-177), one or more drains (e.g., a SET mode drain 178, a fluid in drain 179, a CAL mode drain 180, and four QC drains 181-184, and/or one or more valve drains 185). In some embodiments, the plurality of operating modes can include one or more of a setup mode 1004, an inflow mode 1000, a sense mode (also a ship mode) 1002, a calibration mode 1006, a valve flush mode 1008, and one or more QC modes 1010 (see FIG. 8). As shown in FIG. 8, each of the operating modes can correspond to an open position (marked as “O”) or a closed position (marked as “X”) of each of the inlets or drains on the face valve and the manifold.

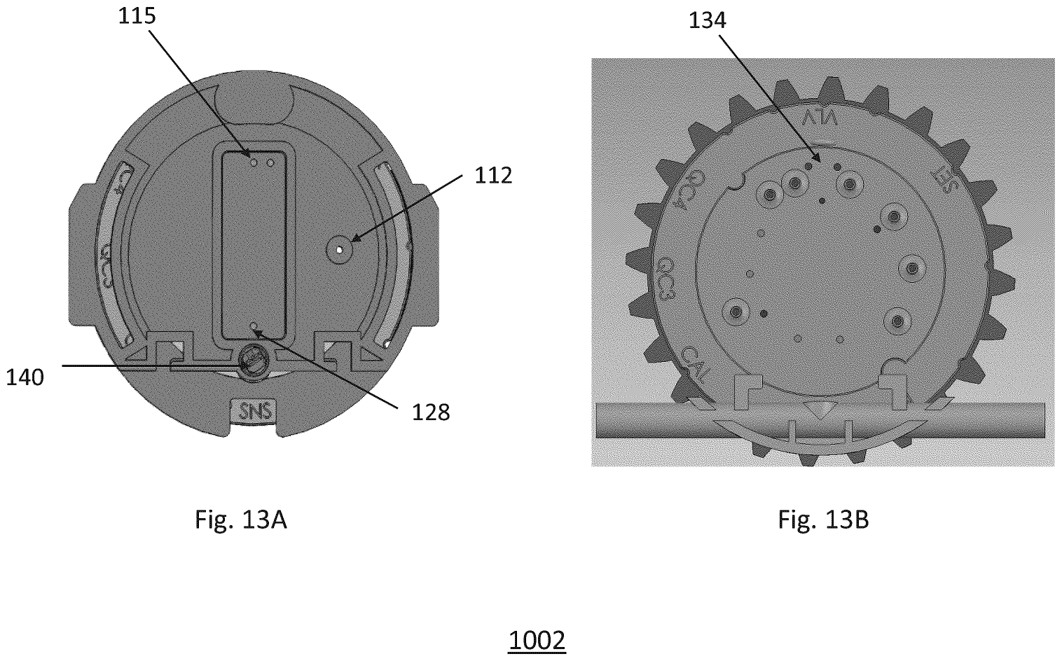

In some embodiments, the fluid sensor cartridge 100 can be shipped and sent to a user in the sense/ship mode 1002 (see FIG. 13A-13B), in which the plug valve 140 can be configured to be closed to the outside environment and all the drains closed to the reservoirs, leaving only a vacuum port 112 open. In some embodiments, the vacuum port 112 can be configured to be either exposed to ambient air or connected to a vacuum source, allowing the fluid sensor cartridge 100 to operate in both negative and positive pressures. For example, the vacuum port 112 can pull from a reservoir filled with air to overcome negative pressure in the sample fluid.

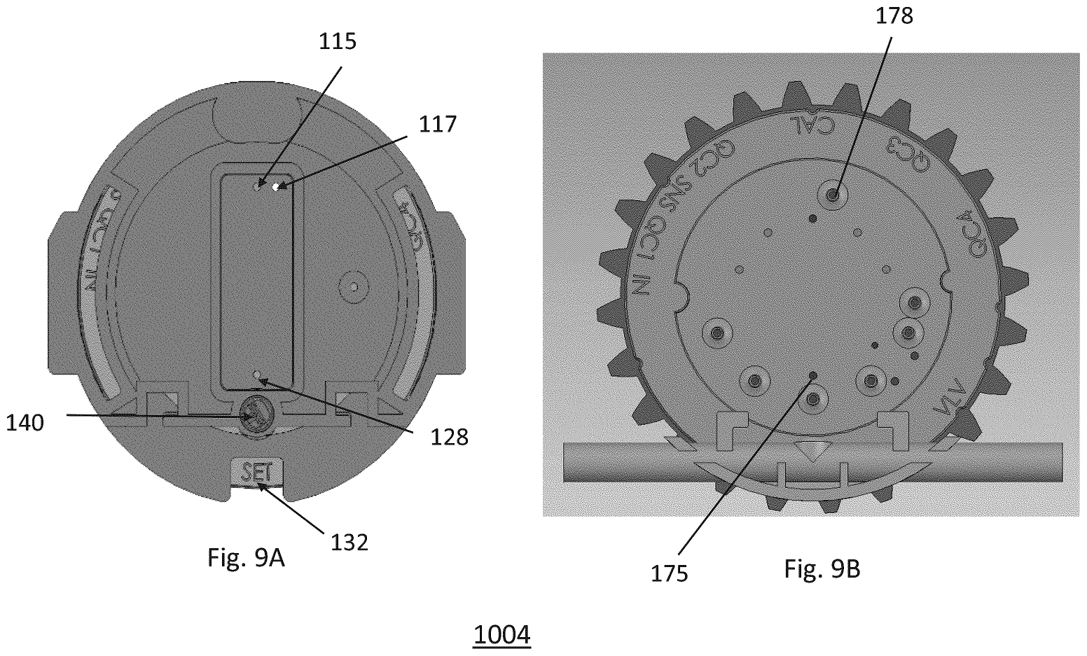

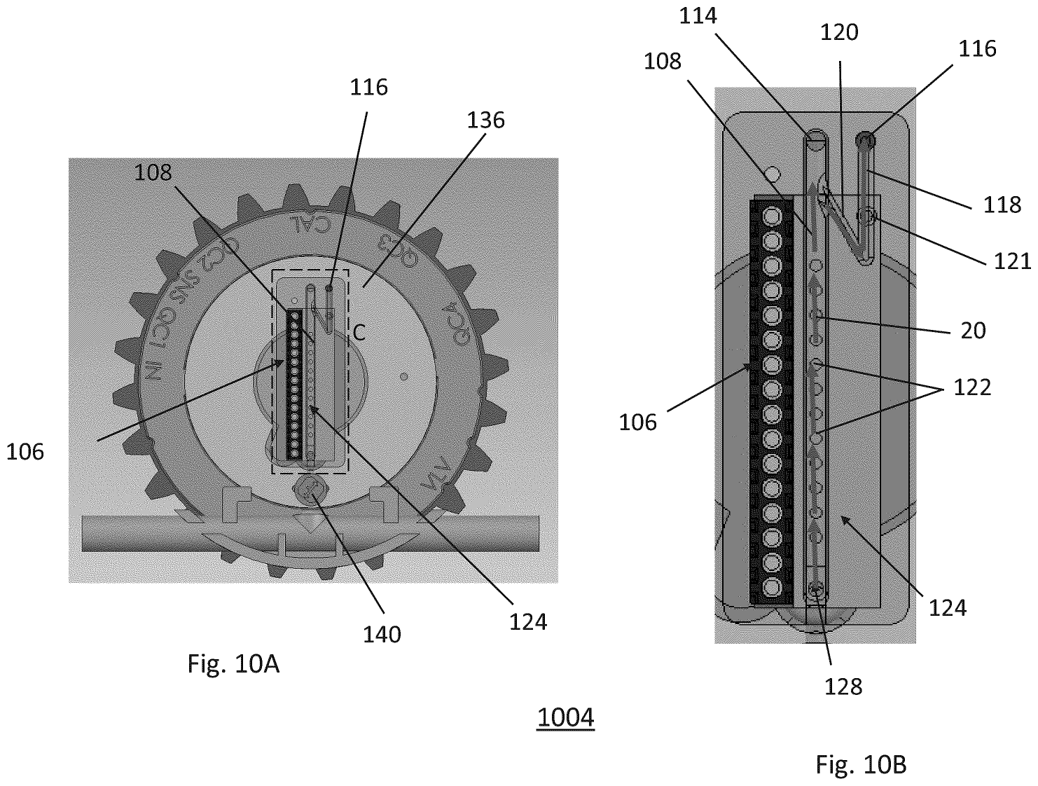

When the fluid sensor cartridge 100 arrives with the user, the user can first operate the fluid sensor cartridge 100 in a setup mode (SET) 1004 as shown in FIG. 9A-10B. In the setup mode 1004, in some embodiments, the calibration fluid inlet opening 128 on the manifold 110 can be configured to be open to the calibration reservoir 155 through the first calibration fluid inlet 175, and a secondary drain hole 117 be configured to open to the waste reservoir 156 via the setup mode drain 178, while the plug valve 140 and other drains remain closed. In this mode, calibration fluid 20 can be purged into the main fluid pathway 108, run over the primary and secondary electrodes 122 and 121, and go to the waste reservoir 156 through the secondary drain hole 117 to the secondary drain 116 and the setup mode drain 178.

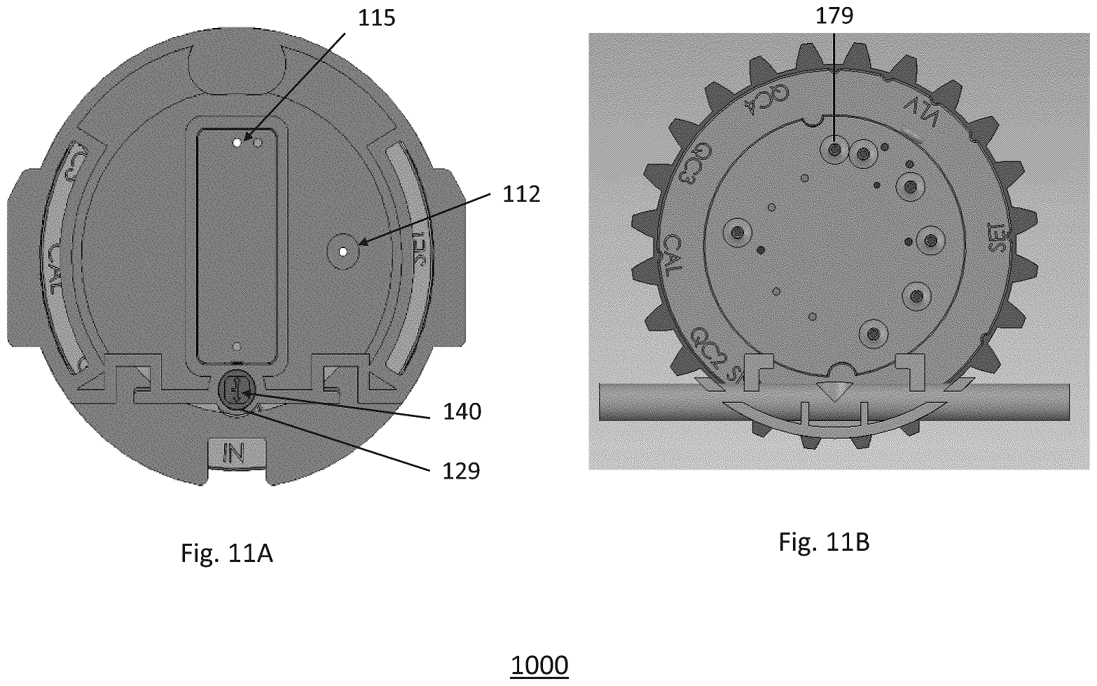

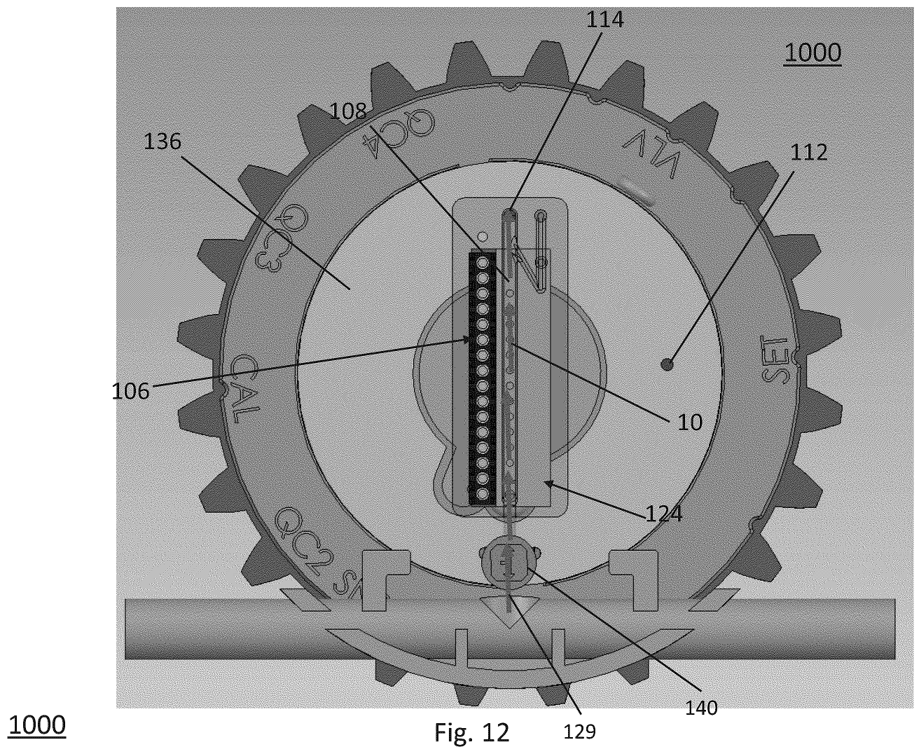

When the fluid sensor cartridge 100 and the patient are ready for a test using the fluid sensor cartridge 100, the user can operate the fluid sensor cartridge 100 in a fluid in or inflow mode (IN) 1000 as shown in FIG. 11A-12. In the fluid in mode 1000, in some embodiments, the plug valve 140 can be configured to be open and connected to the tubing 102 and a primary drain hole 115 be open to the waste reservoir 156 through a fluid in drain 179 on the face valve 130. In this mode, a sample fluid 10 (e.g. patient's blood) can enter the main fluid pathway 108 through the open plug valve 140, run over the primary electrodes 122, and go to the waste reservoir 156 through the primary drain 114. When the sample fluid 10 is in the main fluid pathway 108, in some embodiments, the user can operate the fluid sensor cartridge 100 by rotating both the face valve 130 and the plug valve 140 to stop the sample fluid 10 in the main fluid pathway 108 to achieve the sense mode (SNS) 1002. Therefore, measurement can take place while the sample fluid 10 is stationary in the sense mode 1002.

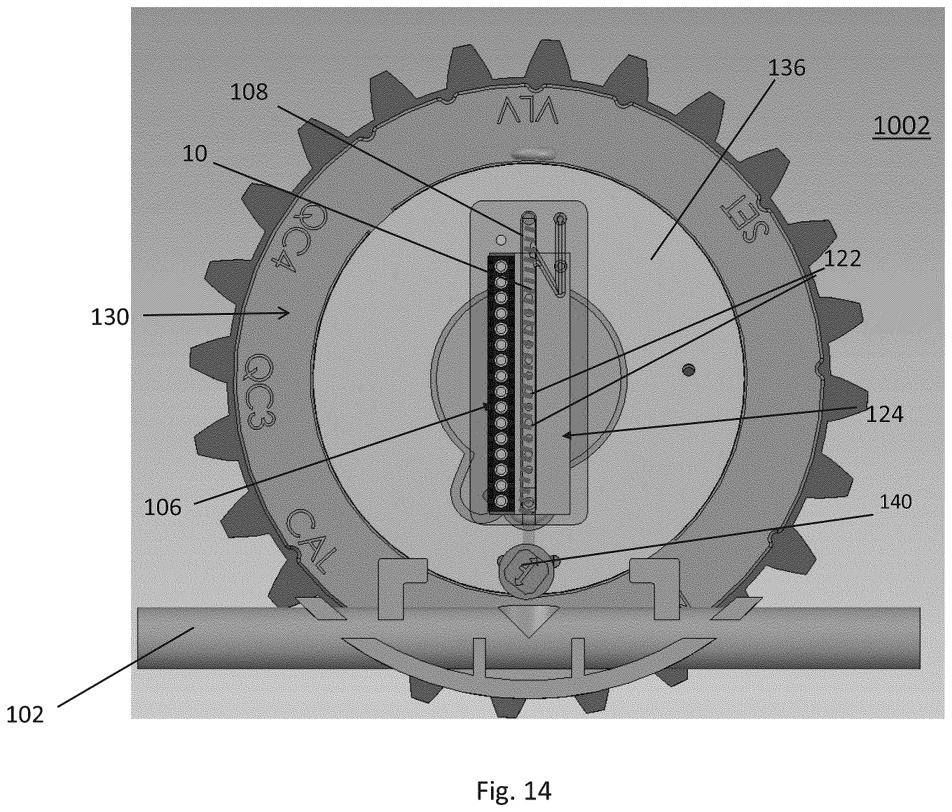

In the sense mode 1002, in some embodiments, the plug valve 140 can be configured to be closed to the outside environment and all the drains closed to the reservoirs as shown in FIG. 13A-14. For example, the primary drain hole 115 can be occluded by the face plate 134 and closed to the waste reservoir 156. In this mode, the sample fluid 10 can stay in the main fluid pathway 108 for a desired period of time, and the primary electrodes 122 be exposed to the sample fluid 10 to sense the constituents in the sample fluid 10.

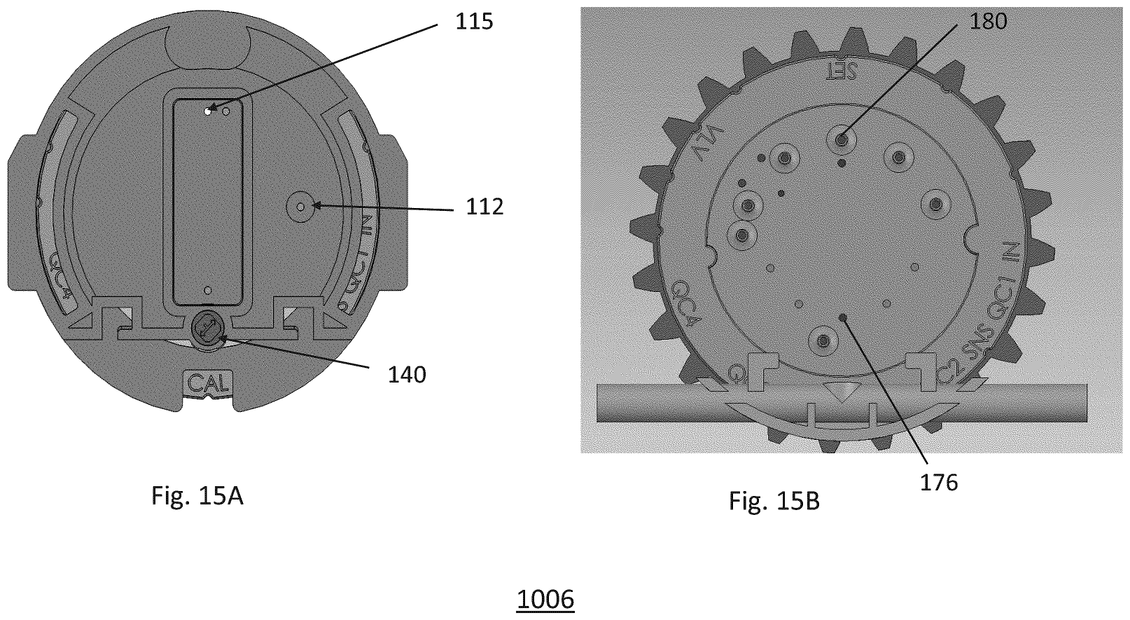

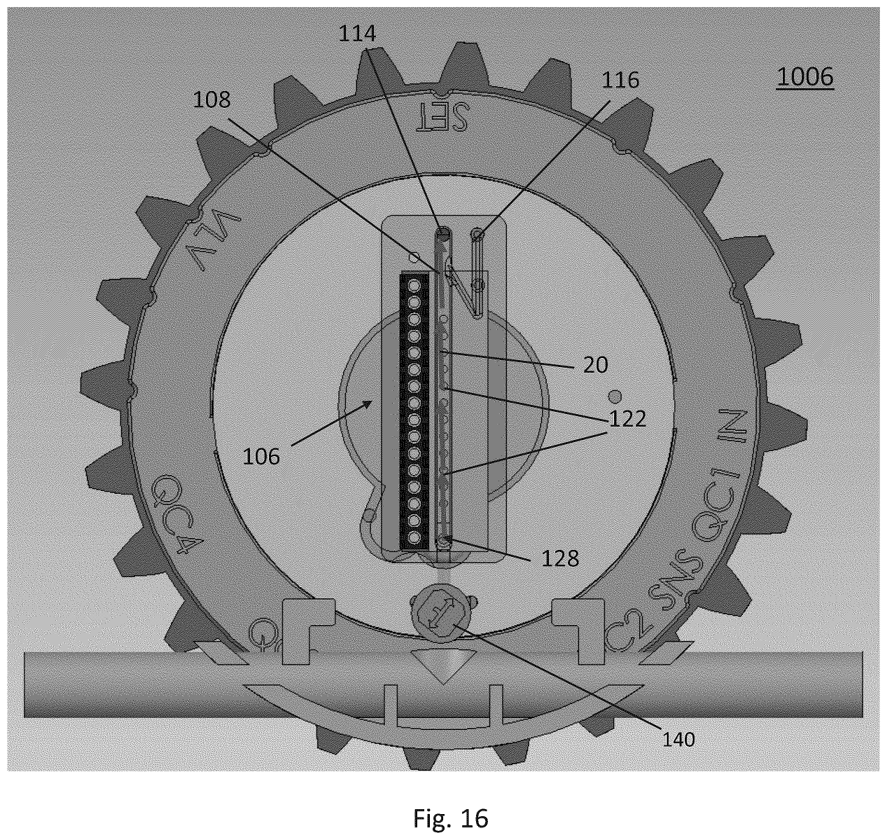

After the sample fluid 10 has run through the main fluid pathway 108 in the fluid in mode 1000 or the sense mode 1002, the main fluid pathway 108 and/or the secondary fluid pathway 119 can be cleaned and the fluid sensor cartridge 100 calibrated in the calibration mode (CAL) 1006 as shown in FIG. 15A-16. In the calibration mode 1006, in some embodiments, the plug valve 140 can remain closed, the face valve 130 is rotated such that the primary drain hole 115 can be open to the waste reservoir 156 through a calibration mode drain 180, and the calibration fluid inlet opening 128 on the manifold 110 be configured to be open to the calibration reservoir 155 through a second calibration fluid inlet 176. In this mode, the calibration fluid 20 can be purged into the main fluid pathway 108, run over the primary electrodes 122, and to the waste reservoir 156 through the primary drain 114. The main fluid pathway 108 and the primary electrodes 122 can thereby be cleaned and reset in the calibration mode 1006.

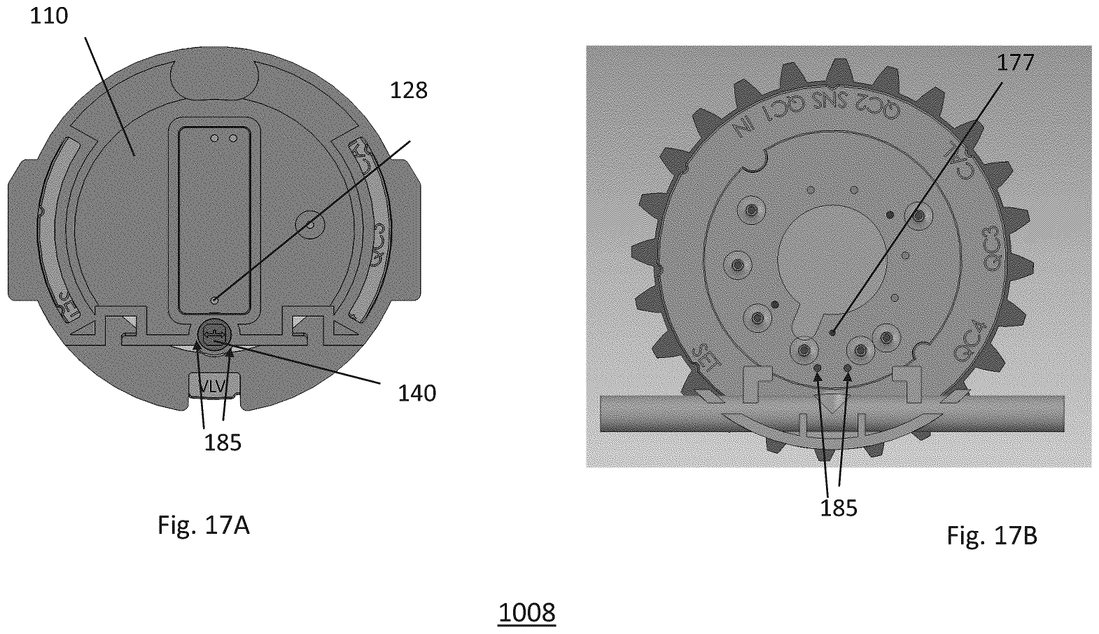

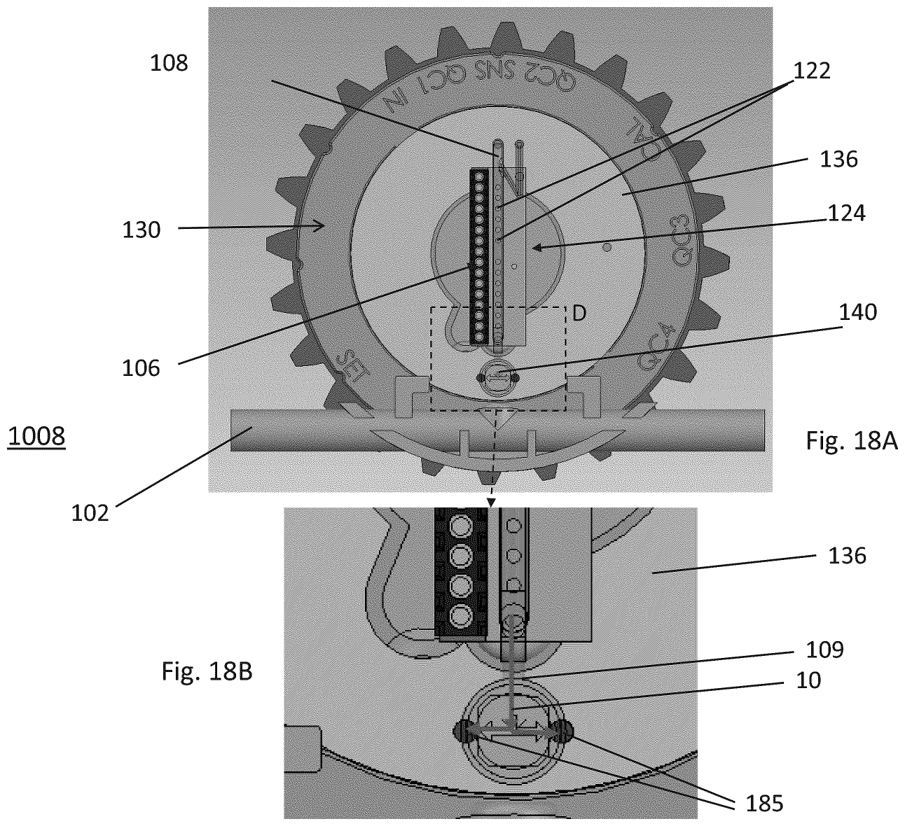

In some embodiments, the plug valve 140 can similarly be flushed and cleaned in the valve flush mode (VLV) 1008 as shown in FIG. 17A-18B. In the valve flush mode 1008, the plug valve 140 is in the open position O in which the plug valve 140 can be configured to be open to the main fluid pathway 108 but closed to the tubing 102. Further, in some embodiments, the plug valve 140 and the face valve 130 can be coordinated such that the plug valve 140 is open to the waste reservoir 156 through a valve flush pathway 109 and the one or more valve drains 185. In this mode, the calibration fluid inlet opening 128 on the manifold 110 can be configured to be open to the calibration reservoir 155 through a third calibration fluid inlet 177, and the primary and secondary drain holes 115 and 117 all be closed so that calibration fluid 20 would only flow towards the plug valve 140.

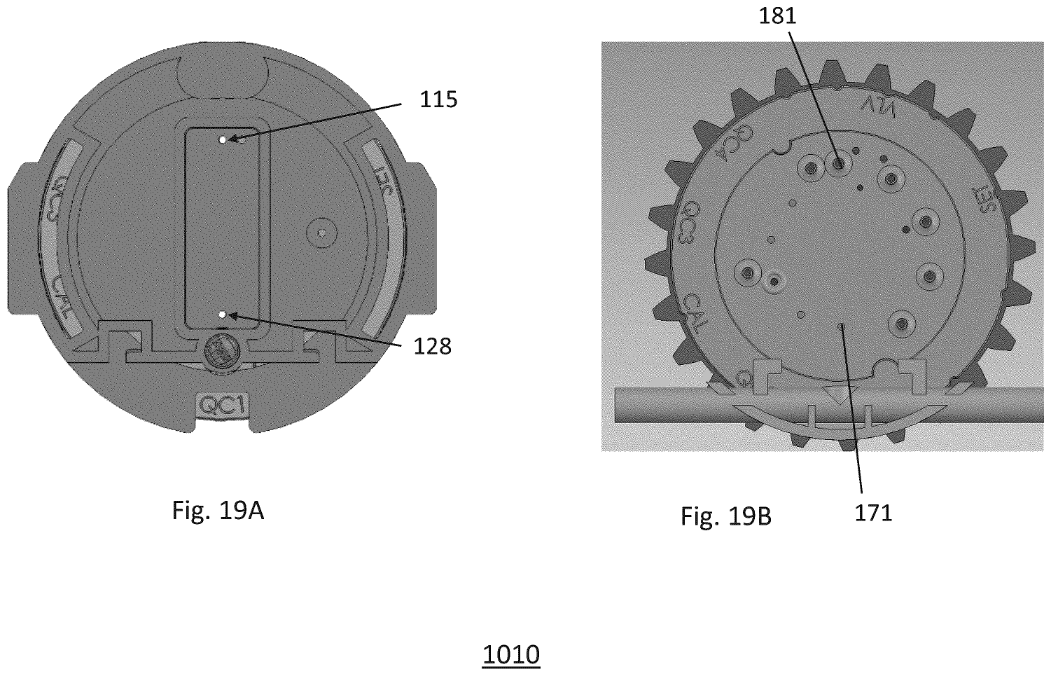

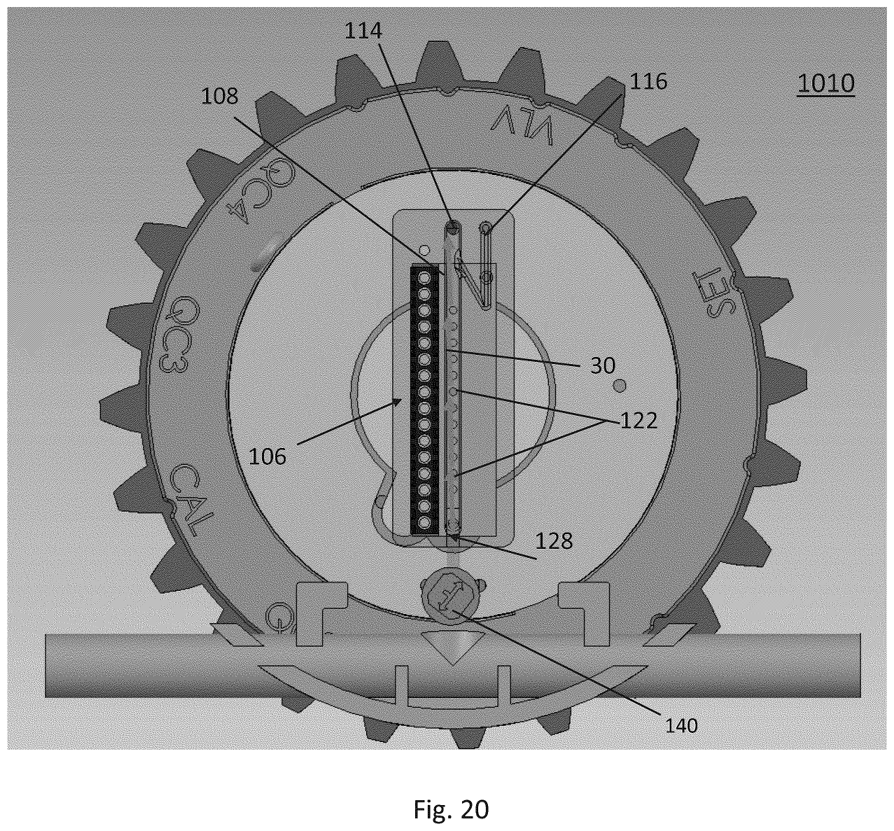

In some embodiments, the fluid sensor cartridge 100 can further include one or more QC modes 1010 to calibrate the sensing elements and maintain quality control of the sensing elements as shown in FIG. 19A-20. In the illustrated embodiment, there are four QC reservoirs 151-154 (see FIGS. 6A-6B) holding four different QC fluids, so there can be four QC modes and four QC inlets 171-174 corresponding to the four QC fluids. As an example, in some embodiments, to calibrate the primary electrodes 122 using the first QC fluid from the first QC reservoir 151, the calibration fluid inlet opening 128 on the manifold 110 can be configured to be open to the first QC reservoir 151 through a first QC inlet 171, and the primary drain hole 115 be configured to be open to the waste reservoir 156 through a first QC drain 181. In this QC mode 1010, the first QC fluid from the first QC reservoir 151 may be purged into the main fluid pathway 108 through the calibration fluid inlet opening 128 on the manifold 110, run through the primary electrodes 122, and go the waste reservoir 156 through the primary drain 114. The sensed data can allow the sensing elements (e.g., the primary electrodes 122) to be electrically calibrated using pre-determined metrices (e.g., concentrations of the QC fluids).

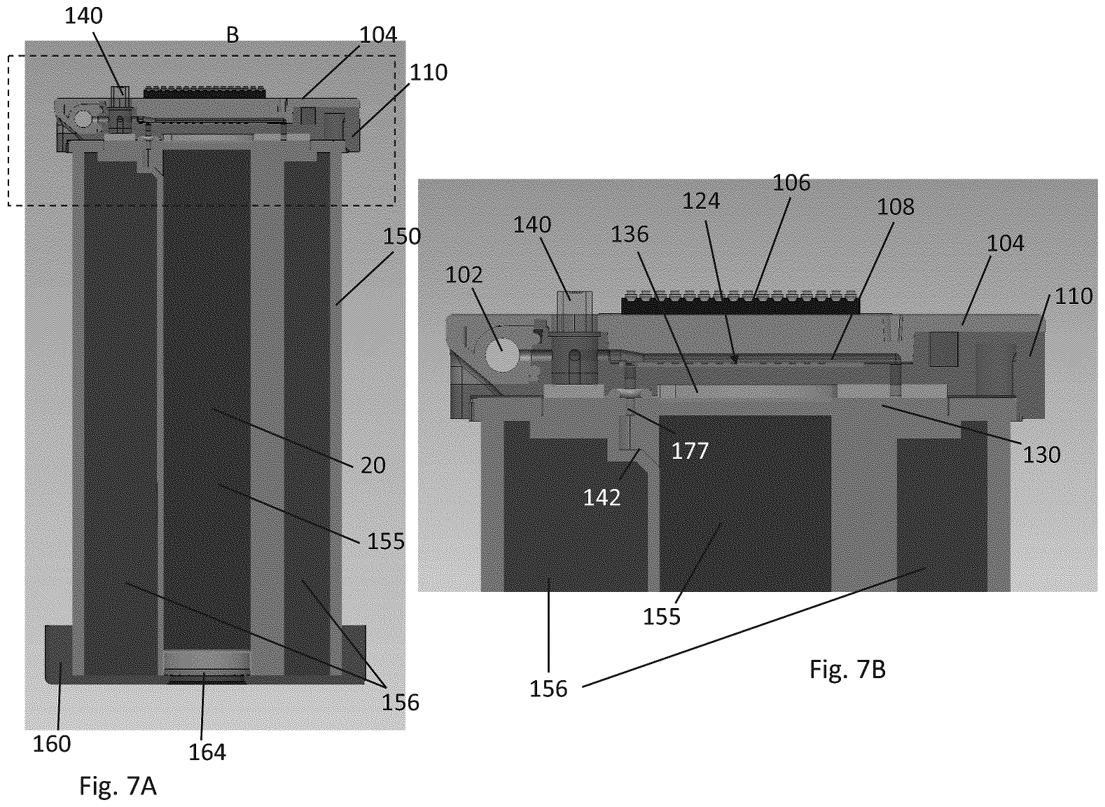

In some embodiments, one or more plungers can be coupled to a bottom end of one or more of the reservoirs to control a flow of the QC or calibration fluid into the main fluid pathway 108. In some embodiments, the one or more plungers can act as a syringe to push or purge the fluids into the main fluid pathway 108. For example, as shown in FIG. 7A, a calibration plunger 164 can be coupled to bottom end of the calibration reservoir 155 and configured to be pushed into the calibration reservoir 155 and purge the calibration fluid 20 up to the main fluid pathway 108 through the calibration channel 142 and the third calibration inlet 177 on the face valve 130. In some embodiments, one or more QC plungers 162 can also be included to purge the one or more QC fluids (FIG. 2B).

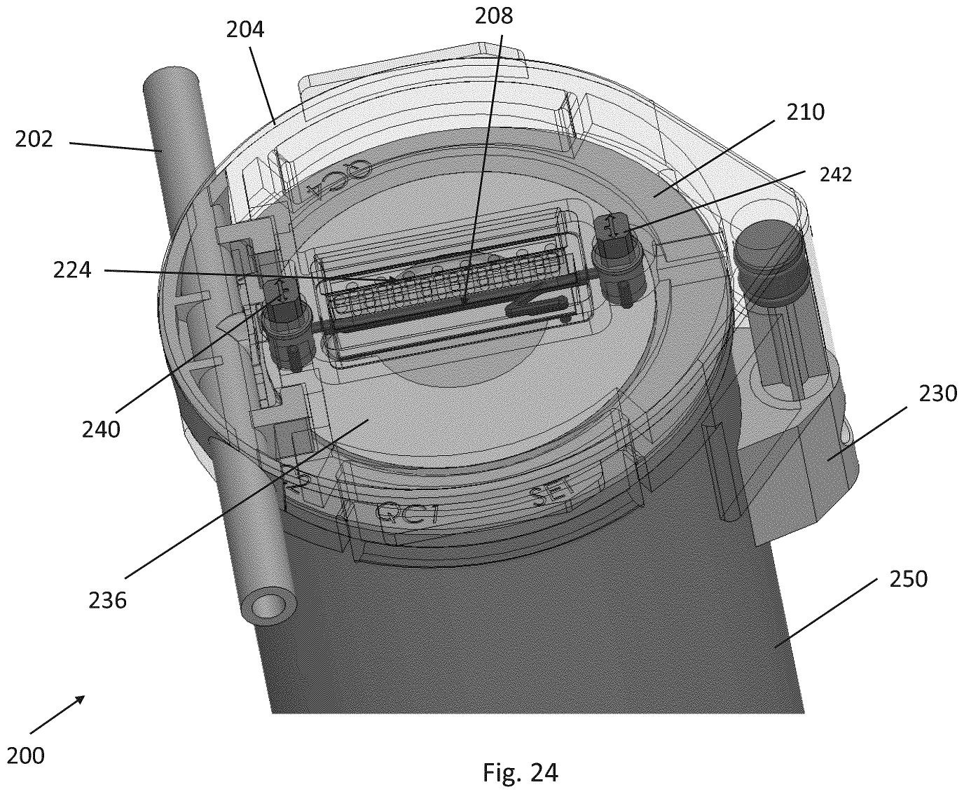

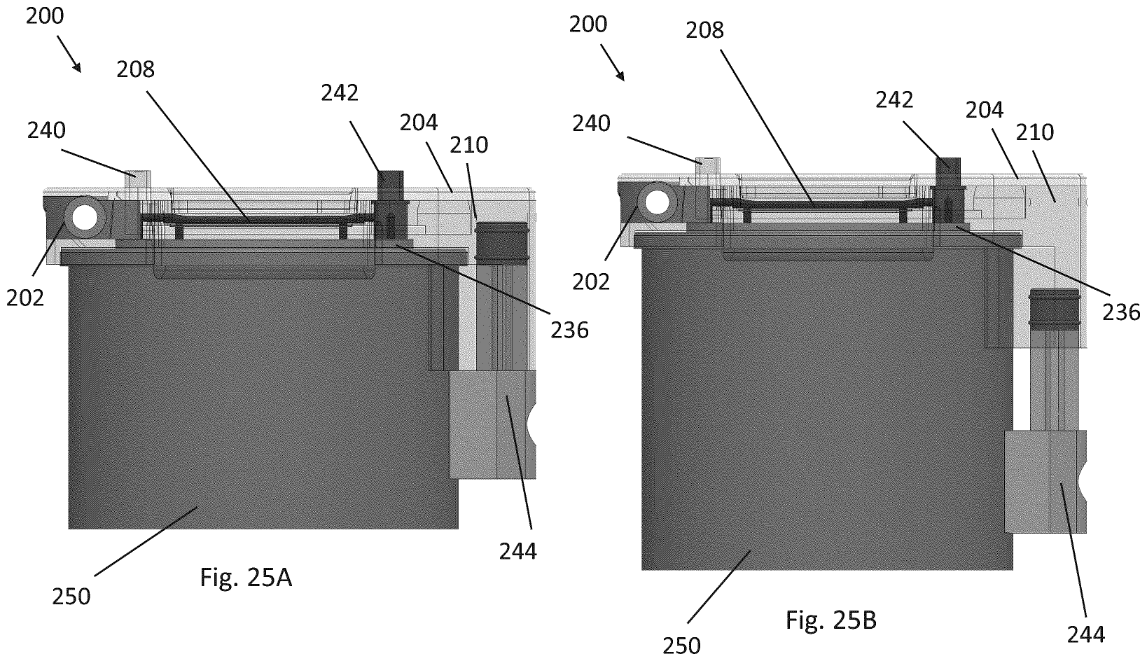

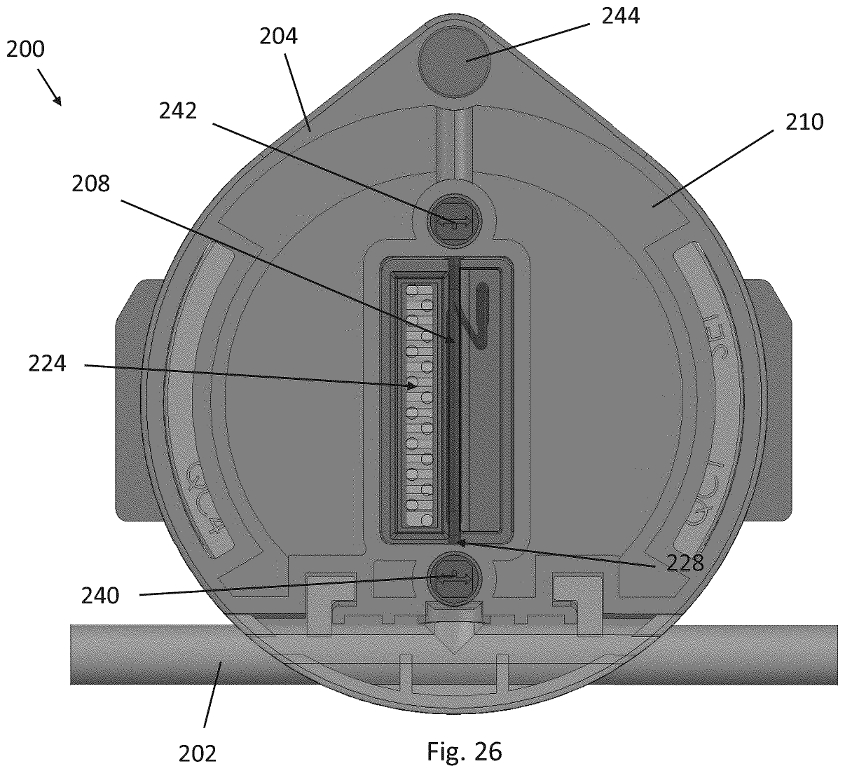

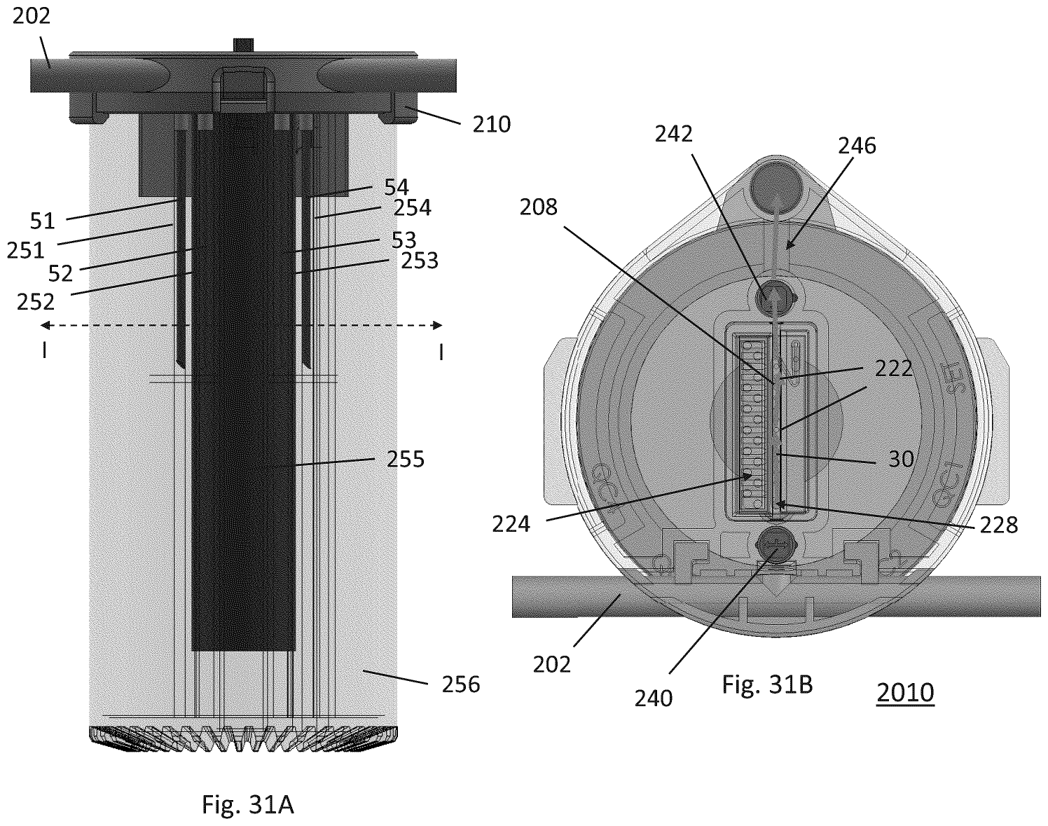

In accordance with various embodiments, to better control the flow and volume of fluids (e.g., calibration fluid, QC fluids, etc.) in a fluid sensor cartridge and simplify the control the motor and controller system, it may be beneficial to include an external plunger 230 that can be fluidly connected to the main fluid pathway 208 in a fluid sensor cartridge 200 as shown in FIG. 24-32C. Unless otherwise noted, elements of the fluid sensor cartridge 200 may have similar functions or operate similarly as similarly-numbered elements of the fluid sensor cartridge 100. In some embodiments, the external plunger 230 can work as a syringe to pull fluids into or out of the main fluid pathway 208 at a certain rate and for a certain volume as needed. In some embodiments, the external plunger 230 can be controllable by hand or by the controller of the reader assembly 190. With the external plunger 230, which may be in direct fluid connection with the main fluid pathway 208, a plurality of fluids can be driven by the same external plunger 230 through their respective inlets or openings, which can overlap with the calibration fluid inlet opening 228 on the manifold 210 by rotating the face valve 230, and through the main fluid pathway 208. In some embodiments, reservoirs without a corresponding plunger directly attached to them can each include a straw so that the external plunger 230 in fluid connection with the fluid pathway can pull fluids from, e.g., the QC reservoirs 251-254 through the straws 51-54 (see FIG. 31A-31B).

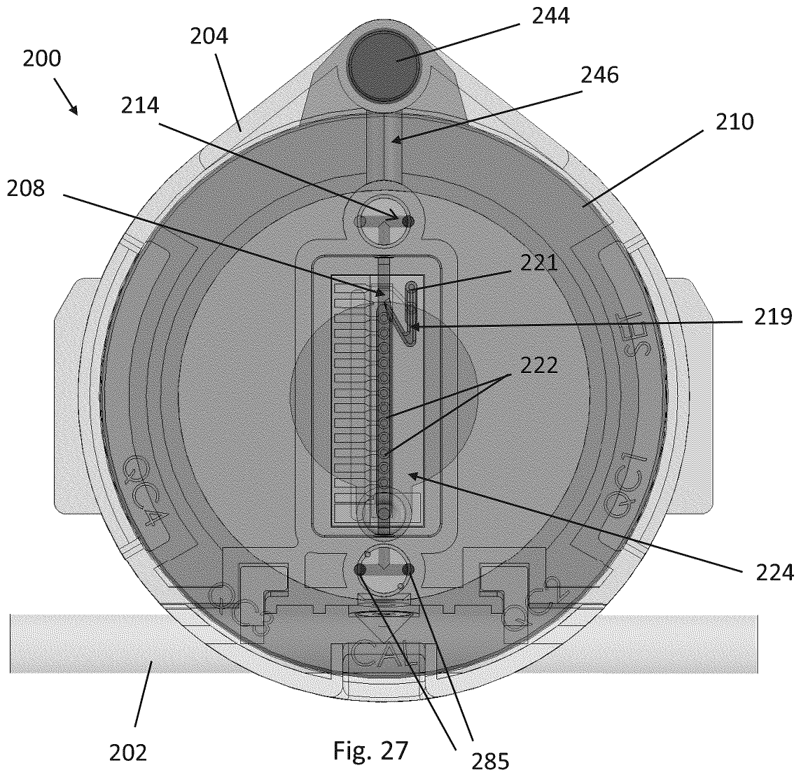

In some embodiments, the fluid sensor cartridge 200 may further include a third valve 242 (e.g. a second plug valve) configured to switch between different positions, allowing the external plunger 230 to control either a fluid in the main fluid pathway 208 or to control only a fluid between the third valve 242 and the external plunger 230. In some embodiment, the third valve 242 can be positioned in an opening of the manifold 210 above the primary drain 214 as shown in FIG. 27, wherein the primary drain 214 can comprise one or more drain openings 215 to align with one or more openings of the third valve 242.

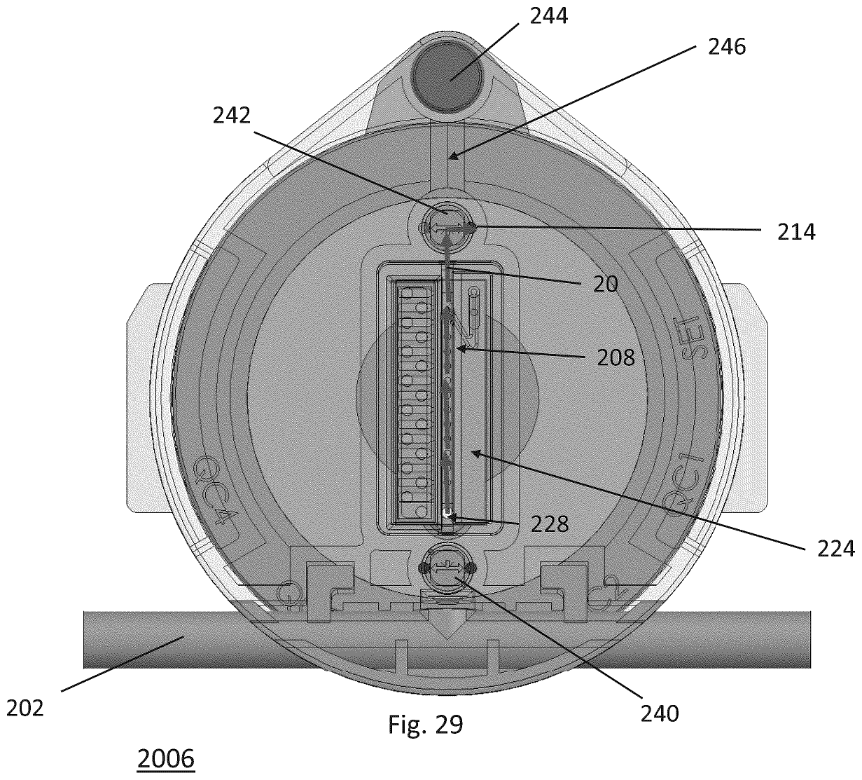

In some embodiments, the sensor fluid cartridge 200 can include additional operating modes with the external plunger 230 and the third valve 242. For example, the third valve 242 can be configured to be closed to the external plunger 230 in a calibration mode 2006, letting calibration fluid 20 be driven up and clean the third valve 242, through the primary drain 214 and to the waste reservoir 256 as shown in FIG. 29.

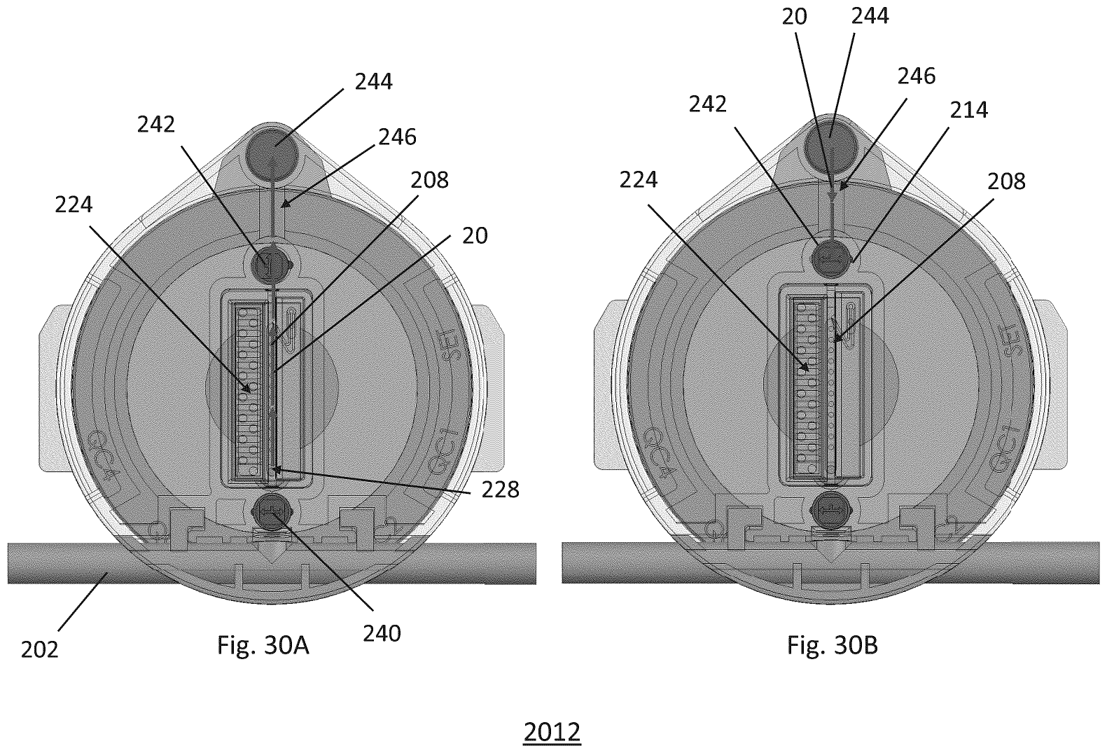

In some embodiments, in a plunger flush mode 2012, the third valve 242 can also be configured to be open to both the main fluid pathway 208 and the external plunger 230 to let the calibration fluid 20 flow through the main fluid pathway 208 and flush the external plunger 230 (see FIG. 30A). After flushing the external plunger 230, in the plunger flush mode 2012, the third valve 242 can be rotated to be closed to the main fluid pathway 208 so that the external plunger 230 can be operated to push the calibration fluid 20 through the primary drain 214 to the waste reservoir 256 (see FIG. 30B).

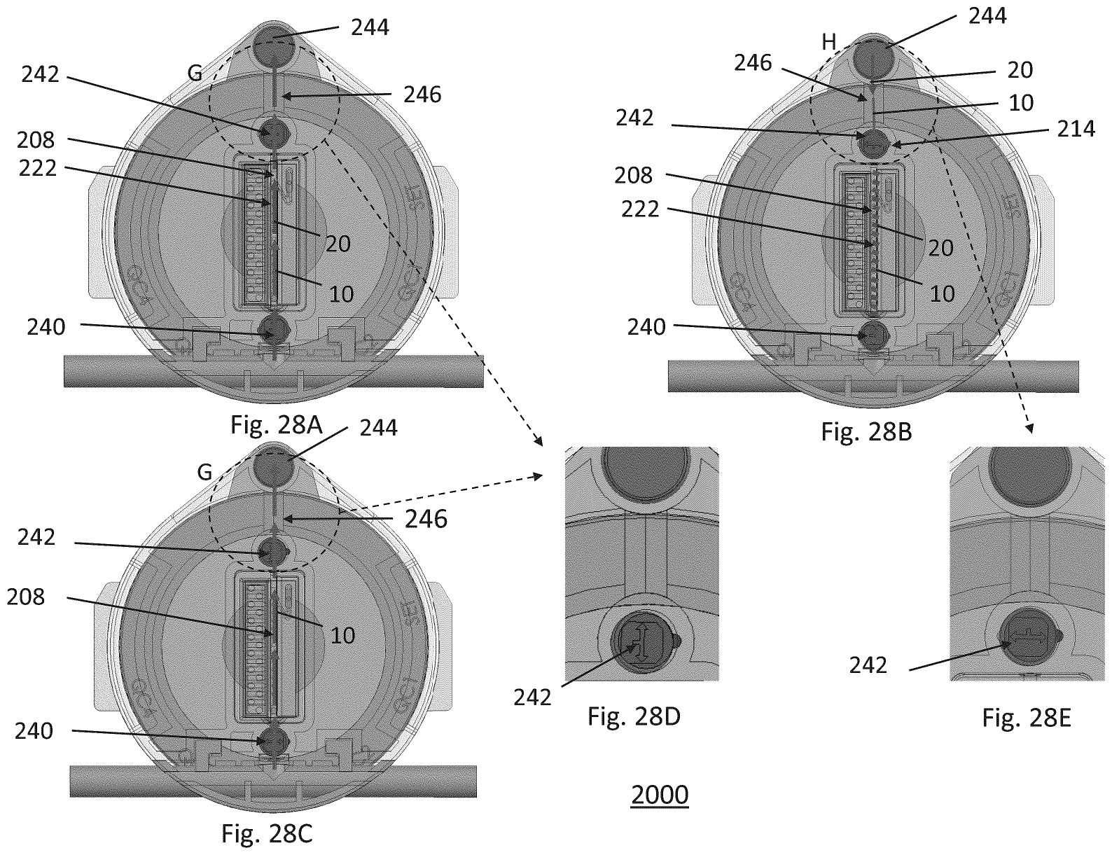

In some embodiments, a fluid in mode (IN or inflow mode) 2000 for the fluid sensor cartridge 200 can operate differently that that for the fluid sensor cartridge 100 with the external plunger 230 and the third valve 242 as shown in FIG. 28A-28E. In the inflow mode 2000, the external plunger 230 can pull the sample fluid 10 through the main fluid pathway 208 with the third valve 242 in an open position O, fluidly connecting the main fluid pathway 208 and the external plunger 230. If the main fluid pathway 208 was previously filled with another fluid, and after pulling the sample fluid 10 into the main fluid pathway 208, the main fluid pathway 208 might be filled with a mixture of the sample fluid 10 and the other fluid (e.g., calibration fluid 20) as shown in FIG. 28A. In this case, the third valve 242 can first be configured to be closed to the main fluid pathway 208 (see FIG. 28B), allowing the external plunger 230 to drive the mixture of fluids through the primary drain 214 to the waste reservoir 256. Then, the third valve 242 can be configured to be open again, allowing the external plunger 230 to drive more sample fluid into the fluid pathway. In some embodiments, this process can be repeated for multiple times until the main fluid pathway 208 is filled solely with the sample fluid 10 and ready for sensing of the sample fluid 10 (see FIG. 28C). Similarly, the QC modes 2010 can implement the same repeated process to ensure the main fluid pathway 208 is not filled with a mixture of different fluid for better quality control.

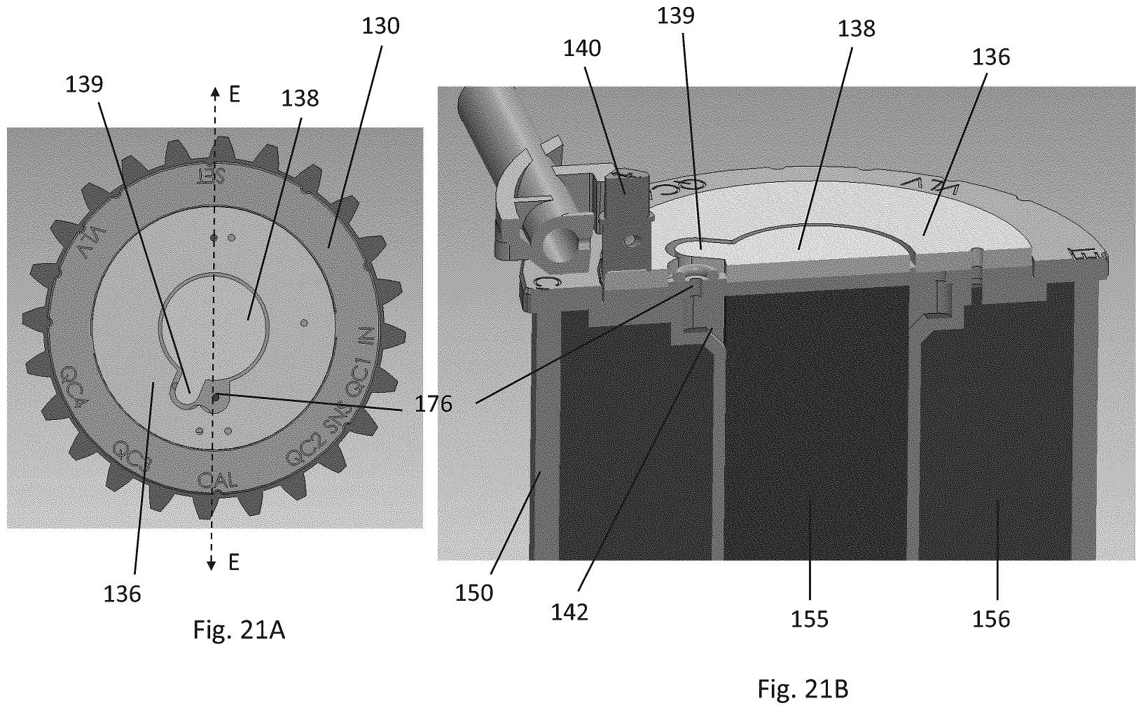

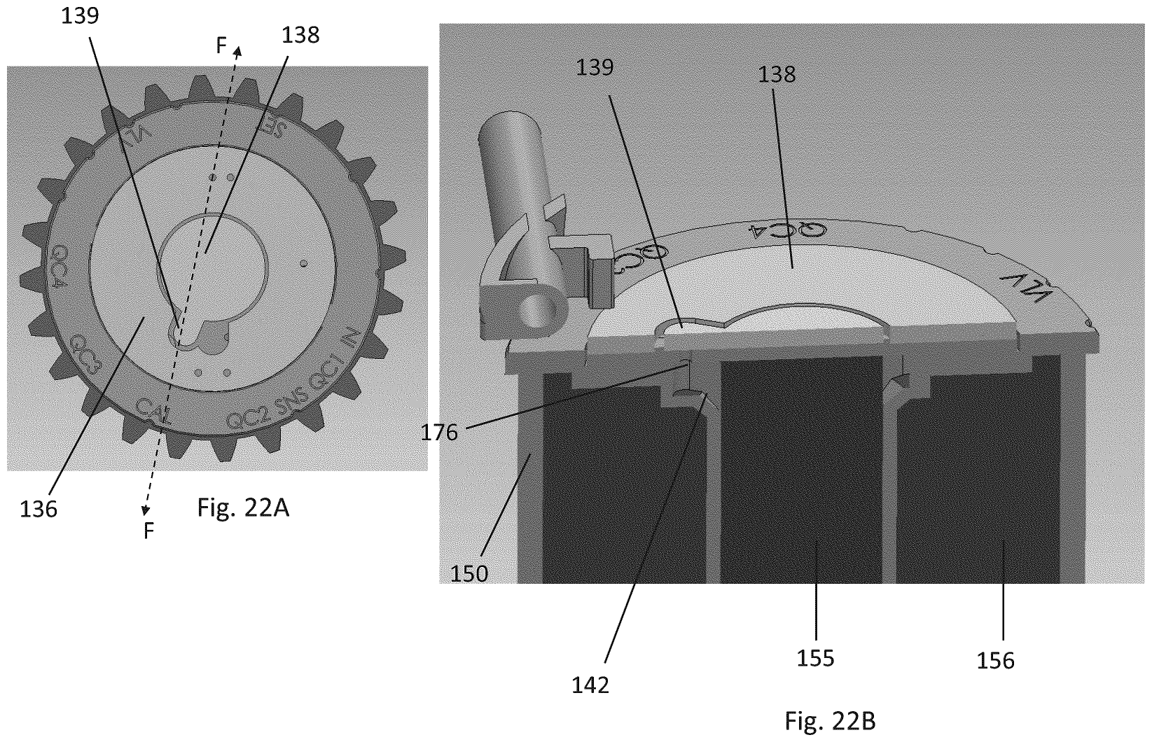

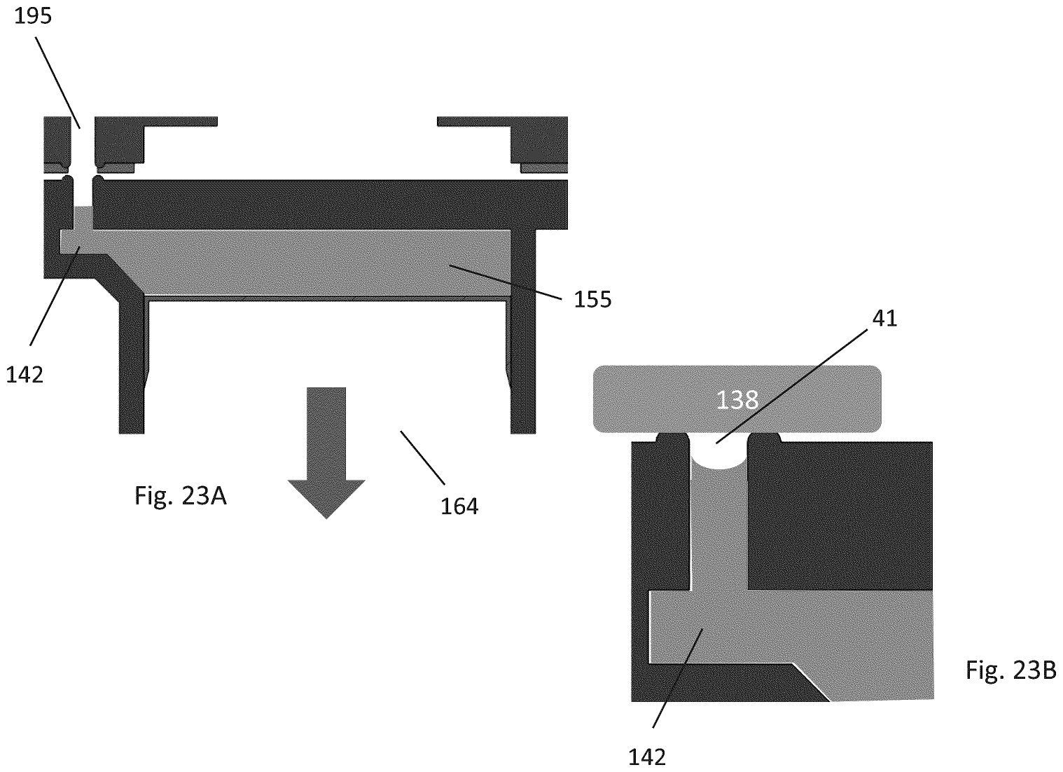

Finally, it can be advantageous to include a mechanism of introducing air bubbles to the fluids (e.g. QC/calibration fluids) so that less amount of fluids would be used to operate the fluid sensor cartridge 100/200 during one or more operating modes. Additionally, air may be introduced to act as a barrier between two different fluids, e.g., when switching between operating modes. In some embodiments, the fluid sensor cartridge 100 can be configured to allow an opening 195 open to the atmosphere to be aligned with the calibration fluid channel 142 and fluidly connected to the calibration reservoir 155 such that pulling the plunger 164 coupled to the calibration reservoir 155 can introduce air to the calibration fluid 20. In some embodiment, a wicking pad 138 positioned between the face valve 130 and the manifold 110, inside the wicking pad 138, can also be used to introduce air bubbles (see FIGS. 21A-22B). For example, in the calibration mode 1006 after sense mode 1002 for the fluid sensor cartridge 100, introduction of air bubbles into the calibration fluid 20 can reduce the amount of the calibration fluid 20 required and more efficiently flush out the sample fluid 10 remains in the main fluid pathway 108. In some embodiments, as shown in FIGS. 21A-23B, air bubbles 41 can be introduced by rotating the face valve 130 to align the calibration fluid channel 142 to a calibration inlet (e.g., the second calibration fluid inlet 176) and to a wicking pad 138 (see FIGS. 22A-22B), purging the calibration fluid 20 out through a calibration fluid channel 142, letting a portion of the calibration fluid 20 to be absorbed by pad projection 139 of the wicking pad 138, and leaving air bubbles 41 in the calibration fluid 20 in calibration fluid channel 142 (see FIG. 23B). In some embodiments, the face valve 130 can then be rotated to align the second calibration fluid inlet 176 to the calibration fluid inlet opening 128 on the manifold 110 on the manifold and allow the calibration fluid 20 having air bubbles to enter the fluid pathway 108.

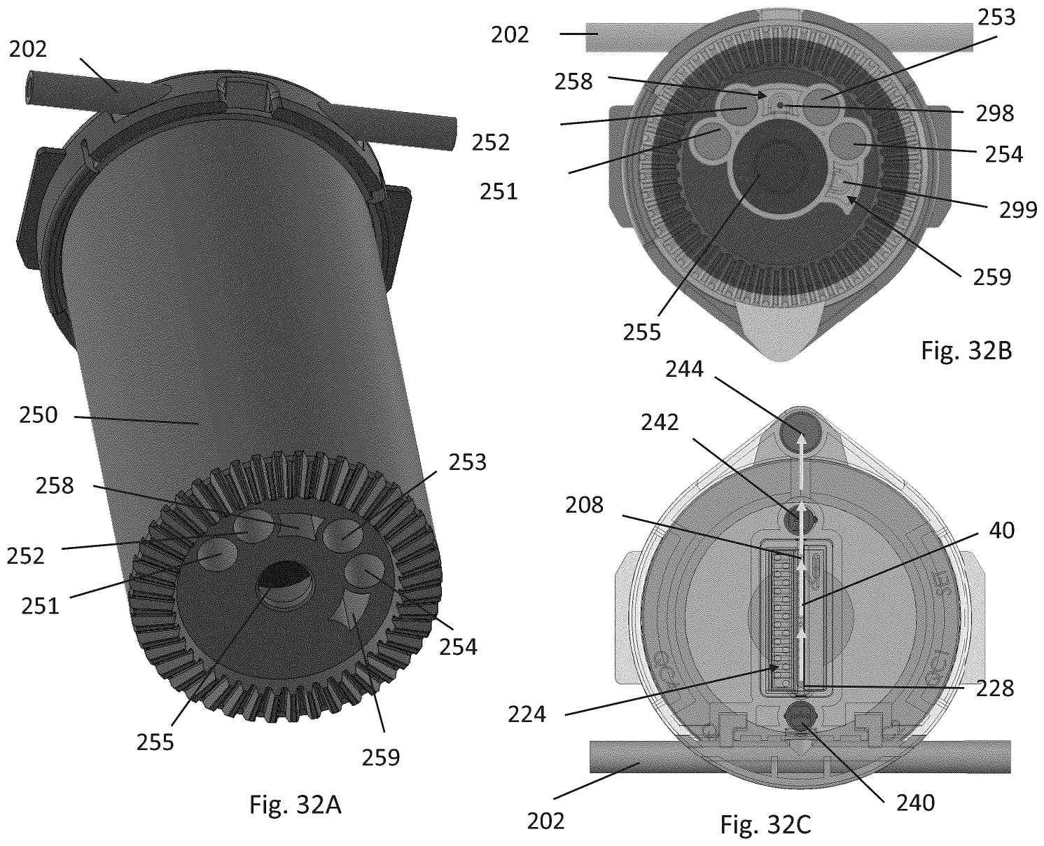

In other embodiments, as shown in FIGS. 32A-32C, the container 250 can include one or more ports exposed to ambient air (e.g., a first air port 258 and a second air port 259). The air ports 258 and 259 can have corresponding air inlet openings (e.g., a first air inlet opening 298 and a second air inlet opening 299) on the face valve 230 that can be configured to overlap with the calibration fluid inlet opening 228 on the manifold 210. For example, in some embodiments, the face valve 230 can be rotated to align a first air inlet opening 298 to the calibration fluid inlet opening 228 on the manifold 210; and the external plunger 230 in fluid connection with the main fluid pathway 208 can be operated to pull air through the calibration fluid inlet opening 228 to the main fluid pathway 208 to introduce air to a fluid or between two different fluids. In some embodiments, the external plunger 230 can also be operated to push air in the main fluid pathway 208 back to the calibration channel 242 to introduce air bubbles into the calibration fluid 20 when needed.

Unless the context clearly requires otherwise, throughout the description and the claims, the words “comprise,” “comprising,” “include,” “including” and the like are to be construed in an inclusive sense, as opposed to an exclusive or exhaustive sense; that is to say, in the sense of “including, but not limited to.” The word “coupled”, as generally used herein, refers to two or more elements that may be either directly connected, or connected by way of one or more intermediate elements. Likewise, the word “connected”, as generally used herein, refers to two or more elements that may be either directly connected, or connected by way of one or more intermediate elements. Additionally, the words “herein,” “above,” “below,” and words of similar import, when used in this application, shall refer to this application as a whole and not to any particular portions of this application. Moreover, as used herein, when a first element is described as being “on” or “over” a second element, the first element may be directly on or over the second element, such that the first and second elements directly contact, or the first element may be indirectly on or over the second element such that one or more elements intervene between the first and second elements. Where the context permits, words in the above Detailed Description using the singular or plural number may also include the plural or singular number respectively. The word “or” in reference to a list of two or more items, that word covers all of the following interpretations of the word: any of the items in the list, all of the items in the list, and any combination of the items in the list.

Moreover, conditional language used herein, such as, among others, “can,” “could,” “might,” “may,” “e.g.,” “for example,” “such as” and the like, unless specifically stated otherwise, or otherwise understood within the context as used, is generally intended to convey that certain embodiments include, while other embodiments do not include, certain features, elements and/or states. Thus, such conditional language is not generally intended to imply that features, elements and/or states are in any way required for one or more embodiments.

While certain embodiments have been described, these embodiments have been presented by way of example only, and are not intended to limit the scope of the disclosure. Indeed, the novel apparatus, methods, and systems described herein may be embodied in a variety of other forms; furthermore, various omissions, substitutions and changes in the form of the methods and systems described herein may be made without departing from the spirit of the disclosure. For example, while elements are presented in a given arrangement, alternative embodiments may perform similar functionalities with different components, and some elements may be deleted, moved, added, subdivided, combined, and/or modified. Each of these elements may be implemented in a variety of different ways. Any suitable combination of the elements and acts of the various embodiments described above can be combined to provide further embodiments. The accompanying claims and their equivalents are intended to cover such forms or modifications as would fall within the scope and spirit of the disclosure.

What is claimed is:

1. A fluid sensor cartridge comprising:
a sensor assembly comprising a fluid pathway and one or more sensing elements in fluid communication with the fluid pathway;
a first fluid inlet configured to provide a first sample fluid to the fluid pathway;
a first valve configured to selectively direct the first sample fluid to the fluid pathway;
a second fluid inlet configured to provide a second fluid to the fluid pathway; and
a second valve configured to selectively direct the second fluid to the fluid pathway.

2. The fluid sensor cartridge of claim 1, further comprising a first drain in fluid communication with the fluid pathway, the first drain to convey fluid from the fluid pathway to a waste reservoir.

3. A fluid sensor cartridge comprising:
a container comprising a calibration fluid reservoir to hold a calibration fluid and a waste reservoir;
a sensor assembly comprising a fluid pathway and one or more sensing elements in fluid communication with the fluid pathway;
a first fluid inlet configured to provide a first sample fluid to the fluid pathway;
a second fluid inlet configured to provide at least a second fluid to the fluid pathway; and
a first drain in fluid communication with the fluid pathway, the first drain to convey fluid from the fluid pathway to the waste reservoir.

4. The fluid sensor cartridge of claim 3, further comprising a first valve configured to selectively direct the first sample fluid to the fluid pathway and a second valve configured to selectively direct the second fluid to the fluid pathway.

5-6. (canceled)

7. The fluid sensor cartridge of claim 3, further comprising a plunger configured to control flow of fluid in the fluid pathway.

8. The fluid sensor cartridge of claim 7, further comprising a third valve configured to fluidly connect the fluid pathway to the plunger.

9. The fluid sensor cartridge of claim 8, wherein the third valve comprises a second rotatable plug valve having a plurality of operating positions.

10. The fluid sensor cartridge of claim 9, further comprising a second drain spaced apart from the first drain, wherein the plurality of openings comprises a setup mode fluid inlet opening and a setup mode drain opening, wherein during a setup mode of the fluid sensor cartridge, the setup mode fluid inlet opening at least partially overlaps with the second fluid inlet, the setup mode drain opening at least partially overlaps with the second drain, and the first drain is occluded.

11-17. (canceled)

18. The fluid sensor cartridge of claim 10, further comprising a third drain to convey fluid to the waste reservoir, wherein the plurality of openings comprises a valve drain opening, wherein in a valve flush mode of the fluid sensor cartridge, the valve drain opening at least partially overlaps the third drain, the third drain disposed below the first valve.

19-34. (canceled)

35. The fluid sensor cartridge of claim 34, further comprising a calibration fluid reservoir in the container and a calibration fluid plunger configured to drive the calibration fluid along a calibration channel of the container and out of the container through a calibration fluid inlet opening of a manifold attached on top of the container.

36-53. (canceled)

54. A fluid sensing method comprising:
opening a first plug valve to provide a first sample fluid to a fluid pathway to expose a sensor assembly to at least the first sample fluid;
transducing at least one fluid property of the first sample fluid with the sensor assembly; and
rotating a second face valve to provide a second fluid to the fluid pathway to flush the sensor assembly, the second fluid flowing along the fluid pathway through a first drain to a waste reservoir.

55. The method of claim 54, further comprising, before transducing, closing the first plug valve and rotating the second face valve to occlude the first drain.

56. The method of claim 54, further comprising rotating the second face valve to provide a third fluid to the fluid pathway, the third fluid flowing along the fluid pathway through the first drain, the third fluid comprising a quality control (QC) fluid.

57. The method of claim 54, further comprising rotating the second face valve to provide a plurality of fluids to the fluid pathway through a plurality of corresponding openings, each of the one or more fluids flowing along the fluid pathway through the first drain.

58. The method of any one of claim 54, further comprising at least partially opening the first plug valve, and rotating the second face valve to provide the second fluid to the first plug valve to flush the first plug valve, the second fluid flowing through the first plug valve through a valve drain to the waste reservoir.

59. The method of any one of claim 54, further comprising rotating a third plug valve to fluidly connect the fluid pathway to a plunger and controlling flow of fluid in the fluid pathway with the plunger.

60. The method of claim 59, further comprising rotating the third plug valve to provide the fluid in the fluid pathway to flow through the third plug valve through the first drain to the waste reservoir.

61. The method of claim 60, further comprising, before transducing and when the fluid in the fluid pathway is a mixture of the sample fluid and another fluid,
closing the third plug valve to the fluid pathway,
controlling flow of the mixture of fluid between the third plug valve and the plunger through the third plug valve through the first drain to a waste reservoir with the plunger,
rotating the third plug valve to fluidly connect the fluid pathway to the plunger,
controlling a flow of sample fluid to the fluid pathway with the plunger,
and repeating the process until the fluid in the fluid pathway comprises substantially of only the sample fluid.

62. The method of claim 60, further comprising rotating the second face valve and rotating the third plug valve to provide the second fluid to the third plug valve to flush the third plug valve and the plunger, the second fluid flowing through the third plug valve to the plunger; and rotating the third plug valve and pushing the plunger to drive the fluid between the third plug valve and the plunger through the first drain to the waste reservoir.

63. The method of claim 60, further comprising pulling air from one or more cavities open to ambient air to introduce air bubbles to fluid in the fluid pathway.
